(19) United States
(12) Patent Application Publication
(10) Pub. No.: US 2013/0079246 A1
(43) Pub. Date: Mar. 28, 2013

(54) Methods and Tools for Screening Agents Exhibiting an Activity on Receptors of the Tumor Necrosis Factor Receptor Superfamily

(71) Applicants: Thibaut De Smedt, Plan-les-Ouates (CH); Laurent Galibert, Plan-les-Ouates (CH); Anne-Renee Van Der Vuurst De Vries, Plan-les-Ouates (CH); Kevin Poupard, Plan-les-Ouates (CH)

(72) Inventors: Thibaut De Smedt, Plan-les-Ouates (CH); Laurent Galibert, Plan-les-Ouates (CH); Anne-Renee Van Der Vuurst De Vries, Plan-les-Ouates (CH); Kevin Poupard, Plan-les-Ouates (CH)

(73) Assignee: ADDEX PHARMA SA, Plan-les-Ouates (CH)

(21) Appl. No.: 13/695,918

(22) PCT Filed: May 5, 2011
(86) PCT No.: PCT/EP2011/057245

§ 371 Date: Nov. 2, 2012

(30) Foreign Application Priority Data
May 5, 2010 (EP) 10162068.0

(51) Int. Cl. C12Q 1/48; C12N 9/96

(52) U.S. Cl. CPC C12Q 1/485; C12N 9/96
USPC 506/10; 435/188; 435/367; 435/361; 435/369; 435/354; 435/364; 435/372.2; 435/372; 435/365; 435/370; 435/352; 435/363; 435/366; 536/23.2

(57) ABSTRACT

The present invention provides novel chimeric receptors and methods of screening using the chimeric receptors. The chimeric receptors comprise an extracellular domain of a tumor necrosis factor receptor superfamily (TNFRSF) receptor and an intracellular domain with kinase activity stemming from a receptor tyrosine kinase. According to an embodiment, the chimeric receptor comprises a full-length TNFRSF receptor. The present invention provides means for screening and testing of modulators of TNFRSF receptors.

### THE FIELD OF THE INVENTION

The present invention relates to the field of drug discovery and drug screening and to the development of assays useful in drug screening. More specifically, the present invention relates to methods of screening agents that are capable of affecting the activity of receptors of the tumor necrosis factor receptor superfamily. The present invention further relates to polypeptides, nucleic acids, vectors and cells, which may be used in such methods.

### BACKGROUND OF THE INVENTION AND PROBLEMS TO BE SOLVED BY THE INVENTION

As currently practiced in the art, drug discovery is a long and multiple step process involving identification of specific disease targets, development of an assay based on a specific target, validation of the assay, optimization and automation of the assay to produce a screen, high-throughput screening (HTS) of compound libraries using the assay to identify “hits”, hit validation and hit compound optimization. The output of this process is a lead compound that goes into pre-clinical and, if validated, eventually into clinical trials. In this process, the screening phase is distinct from the assay development phases, and involves testing compound efficacy in living biological systems.

The conventional measurement in early drug discovery assays used to be radioactivity. However, the need for more information, higher throughput and miniaturization has caused a shift towards using fluorescence and/or luminescence detection. Fluorescence-based reagents can yield more powerful, multiple parameter assays that are higher in throughput and information content and require lower volumes of reagents and test compounds. Fluorescence is also safer and less expensive than radioactivity-based methods. Automatized fluorescence plate readers (FLIPR) have been extensively used in the context of drug discovery to measure fluorescence in the context of HTS. In particular, fluorescence-based, quantitative reliable and time-resolved HTS methods have been developed for chemical active agents of G-protein coupled receptors (GPCRs).

However, for tumor necrosis factor receptor superfamily (TNFRSF), dynamic and quantitative drug screen systems have not yet been established. Signalling TNFRSF members are characterized by an extracellular N terminal region including one to six cysteine-rich domains (CRDs), an intracellular C terminus and a single hydrophobic transmembrane spanning domain. The problems of providing efficient screening systems with these types of receptors may be associated with the particular mechanisms and interactions involved in ligand binding and signal transduction, generally requiring oligomerization of the receptor and often involving oligomerized ligands.

A TNFRSF member's signaling process is initiated through trimerization of the extracellular domains of the receptor molecules by the corresponding (and inherently trimeric) cognate ligand of the TNF superfamily (TNFSF) (Bodmer et al. 2002 Trends Biochem. Sci. 27, 19).

Ligand-induced receptor trimerization has been demonstrated by structure analysis obtained after co-crystallization of TNF-β (TNFSF1) and TNFR1 (TNFRSF1A) (Banner et al 1993 Cell 73:431). This trimeric scaffold is believed to be conserved between all TNFSF members including death-domain (DD) containing and TNF receptor associated factor (TRAF) interacting TNFRSF subgroups. Therefore, ligand-induced trimerization of surface receptor chains is now seen as a common initiating event in the TNFRSF signaling cascades (Singh et al. 1998 Prot. Sci. 7:1124, Mongkolsapaya et al. 1999 Nat Struc Biol 6:1048, Kanakaraj et al. 2001 Cytokine 13:25, Oren et al. 2002 Nat Struc Biol 9:288).

Trimerization of the extracellular domains brings the intracellular domains of the three receptor molecules into proximity, which may then be optimally recognized by cytoplasmic adaptor proteins such as TNF receptor associated factor 2 (TRAF2), TNF receptor type 1-associated death domain protein (TRADD), or receptor interacting protein (RIP). Both crystal structure analysis and modeling experiments revealed that, like TNFSF ligands, TRAF2 is assembled into trimers when recruited to TNFRSF members (McWhirter et al. 1999 Proc Natl Acad Sci 96:8408, Park et al. 1999 Nature 398:533). Thus, trimerization is a central event in TNFRSF signal transduction as it applies to both the extracellular receptor-ligand interaction and to the downstream intracellular signalosome architecture.

Functional studies revealed that assembly of ligand-receptor trimers into higher complexity structures (n-trimers) might be required for optimal signal transduction by TNFRSF members from both the DD-containing receptor group and the TRAF interacting receptor group (Holler et al. 2003 Mol Cell Biol. 23(4):1428, French et al. 2005 Blood 105:219, Miconnet 2008 Vaccine 26:4006). The structural basis for assembly of trimers into n-trimers (hexa-, nona-, dodecamers, etc) is thought to rely on the fact that, independently of TNFSF ligand expression, receptor subunits can self-associate through 1) intermolecular disulfide bonding (i.e. TNFRSF5 and 7), and/or 2) by non-covalent interactions implicating the TNFRSF members N-terminal CRD, also called the pre-ligand assembly domain (PLAD) (Chan 2007 Cytokine 37:101).

Thus, in the absence of ligand, a number of TNFRSF members exist in the form of homodimers. Upon ligand engagement, each pre-assembled dimer has the capacity to engage two trimeric ligands and therefore may form molecular bridges between trimers leading to receptor trimers aggregation.

Tumor necrosis factor (TNF), the natural ligand of tumor necrosis factor receptor 1 and 2 (TNFR1 and TNFR2/TNFRSF1B, respectively), plays a central in the pathogenesis of inflammatory diseases and neutralizing monoclonal antibodies against TNF (such as Infliximab and Adalimumab) or soluble TNFR-immunoglobulin fusion proteins (such as Etanercept) have been successfully used in the treatment of diseases such as rheumatoid arthritis, ankylosing spondylitis, psoriasis, and psoriatic arthritis.

As detailed further below, abnormal levels of TNFSFs have been shown to be implicated in many disorders and disease conditions,and thus there is an interest in developing an assay allowing for quantitative and dynamic HTS of agents exerting an activity on receptors of this family.

As of today, proteins constitute the only therapeutic modality for targeting TNFRSFs. Protein therapeutics have drawbacks such as route of administration (they are injectables), high cost of production, and development of antibodies, among others (Semin Cutan Med Surg. March 2007; 26(1):6-14). There is therefore a need to identify alternate and improved drugs, such as small molecule inhibitors, for the treatment of disorders involving TNFSFs. Small molecules present the advantage of being orally available with convenience of use and increased patient compliance, non-immunogenicity, and lower manufacturing costs. Small molecules have also the potential to cross the blood-brain barrier and treat pathologies of the central nervous system (CNS) otherwise not accessible to large proteins such as antibodies and recombinant receptors.

Several prior art assays exist at present to allow monitoring of TNFRSF activity, stimulation and/or levels. For example:
Monitoring modulation of TNFRSF member trafficking and the assembly of complex I by cell fractionation and immunoprecipitation;
Monitoring the formation and activation of the IκB kinase (IKK) complex by Western blotting, ubiquitination and kinase assays;
Monitoring the phosphorylation and degradation of IκB by Western blotting;
Monitoring activation of NF-κB by immunofluorescence techniques;
Monitoring translocation of NF-κB from cytoplasm to nucleus by immunofluorescence techniques;
Monitoring the transcriptional NF-κB activity by luciferase reporter assays;
Monitoring production of TNF-induced NF-κB target genes such as Interleukin-1, Interleukin-6 or Interleukin-8 by ELISA;
Monitoring cytotoxicity and cell death by activation of caspases.

However, these methods have several drawbacks: They measure events distal to the target receptor, and/or they are cumbersome and not amenable to HTS, and/or they do not measure target-specific events. In general, these prior art methods are not suitable for rapid, dynamic and quantitative HTS.

The present invention addresses the problems indicated above. In particular, the present invention addresses the problem of providing an efficient system allowing for rapid, dynamic and quantitative HTS of active agents of TNFRSF members. It is in particular an objective to provide a non-invasive and/or non-destructive method of screening, which allows monitoring cells exposed to candidate compounds over desired time intervals.

It is another objective to provide a way allowing the identification of novel treatments of conditions and diseases related to receptors of the TNFRSF and/or their ligands and/or conditions and diseases that can be improved by acting on such receptors.

Bernard et al. (1987). Proc. Natl. Acad. Sci. USA 84, 2125-2129 disclose a chimeric receptor containing the extracellular interleukin-2 (IL-2)-binding portion of the human IL-2 receptor and the transmembrane and intracellular domains of the human EGF receptor. This chimeric receptor was not functional as it did not lead to autophosphorylation of the chimeric receptor in the presence of the ligand, a feature that is required for the release of free calcium to the cytoplasma. Moreover, this study did not relate to drug discovery and the results of the study would not suggest that the chimeric receptors could be useful in screening methods. Furthermore, the IL-2 receptor is a receptor that does not belong to the currently 29 members of the TNFRSF.

The objectives and problems as discussed above are part of the present invention, and further objectives and solutions become apparent from the more specific description of the invention below.

### SUMMARY OF THE INVENTION

Surprisingly, the present inventors showed that artificial proteins resulting from the fusion of a tumor necrosis factor receptor superfamily (TNFRSF) receptor, or at least the extracellular, ligand-binding portion thereof, with at least the intracellular, kinase portion of a receptor tyrosine kinase (RTK) can be expressed in host cells. Surprisingly, ligand engagement to such chimeric receptors can transduce RTK-like signals, such as the release of free calcium to the cytoplasm, for example. Remarkably, the generated RTK-like signal can be measured in a dynamic, time-resolved, qualitative and quantitative manner in HTS.

According to an aspect, the invention provides a chimeric and/or fusion polypeptide comprising:
a first part comprising an extracellular, ligand-binding portion of a receptor A, said receptor A being selected from TNFRSF receptors;
a second part comprising an intracellular, signalling kinase portion of a receptor B, said receptor B being selected from RTKs; and,
a third part comprising a transmembrane domain.

According to an aspect, the invention provides a chimeric and/or fusion polypeptide comprising:
a first part comprising an amino acid sequence of an extracellular, ligand-binding portion of a receptor A, said receptor A being selected from TNFRSF receptors;
a second part comprising an amino acid sequence of an intracellular, signalling kinase portion of a receptor B, said receptor B being selected from RTKs; and,
a third part comprising an amino acid sequence of a transmembrane domain.

According to an aspect, the present invention provides a chimeric and/or fusion polypeptide comprising:
a first part comprising an amino acid sequence that is taken from and/or substantially identical to the amino acid sequence of a full-length amino acid sequence of a receptor A or at least of an extracellular, ligand-binding portion thereof, wherein said receptor A is selected from TNFRSF receptors;
a second part comprising an amino acid sequence taken from and/or substantially identical to the amino acid sequence of an intracellular, signalling kinase portion of a receptor B, said receptor B being selected from RTKs; and,
if not comprised in said first or said second part, between said first and second parts, a third part comprising an amino acid sequence taken from and/or substantially identical to a transmembrane domain, and/or,
between said first and second parts, a part comprising an amino acid sequence taken from and/or substantially identical to a death domain.

In an aspect, the present invention provides a chimeric polypeptide comprising:
a first part comprising an amino acid sequence that is substantially identical to an extracellular, ligand-binding portion of a receptor A, said receptor A being selected from receptors of the tumor necrosis factor receptor super family (TNFRSF);
a second part comprising an amino acid sequence that is substantially identical to an intracellular, signalling kinase portion of a receptor B, said receptor B being selected from receptor tyrosine kinases (RTKs); and,
between said first and second parts, a third part comprising an amino acid sequence taken from and/or substantially identical to a transmembrane domain.

In an aspect, the present invention provides a chimeric and/or fusion polypeptide comprising:
an amino acid sequence that is substantially identical to the amino acid sequence of the extracellular, ligand binding portion of a receptor A, said receptor A being selected from receptors of the TNFRSF,
a transmembrane domain;
optionally, an amino acid sequence that is substantially identical to the amino acid sequence of a death domain; and,
an amino acid sequence that is substantially identical to the amino acid sequence of an intracellular, signalling kinase portion of a receptor B, said receptor B being selected from receptor tyrosine kinases (RTKs).

According to an aspect, the invention provides a chimeric and/or fusion polypeptide comprising at least:
an extracellular, ligand-binding portion of a TNFRSF receptor;
a transmembrane domain, and,
an intracellular, signalling kinase portion of an RTK.

In an aspect, the present invention provides a method of screening active agents in general, but preferably of a receptor A selected from TNFRSF receptors, said method comprising the steps of:
providing cells expressing at least one nucleotide sequence encoding the chimeric polypeptide of any one aspect of the present invention;
exposing a candidate agent to be screened to said cells;
measuring a physical, biological and/or chemical value that is associated with and/or corresponds to a cellular condition of said cells; and
determining, from the value measured in the preceding step, if said candidate agent is an agent exerting an activity on said receptor A.

In a aspect, the present invention provides a method of screening active agents, preferably of a receptor A selected from receptors of the TNFRSF, said method comprising the steps of:
providing cells expressing at least one nucleotide sequence encoding and/or cells containing the chimeric polypeptide of the invention;
exposing a candidate agent to be screened to said cells;
measuring a physical, biological and/or chemical value that is associated with and/or corresponds to a cellular condition of said cells; and
determining, from the value measured in the preceding step, if said candidate agent is an active agent of said receptor A.

In an aspect, the present invention provides method of screening agents, which are capable of affecting the activity of a receptor A selected from receptors of the tumor necrosis factor receptor super family (TNFRSF), said method comprising the steps of:
providing cells expressing at least one nucleotide sequence encoding the chimeric polypeptide of the invention;
exposing a candidate agent to be screened to said cells;
measuring a physical, biological and/or chemical value that is associated with a cellular condition of said cells; and
determining, from the value measured in the preceding step, if said candidate agent is an agent exhibiting an activity on said receptor A.

In further aspects, the present invention provides nucleic acids comprising one or more nucleotide sequences encoding any one of the chimeric polypeptides according to the invention, one or more transcription vectors comprising one or more nucleotide sequences encoding any one of the chimeric polypeptides according to the present invention, cells expressing any one of the nucleotide sequences of the invention, cells comprising one or more transcription vectors as defined herein, cells containing any one of the chimeric polypeptides of the invention and cells in a membrane of which is embedded any one or more of the chimeric polypeptides of the invention.

In an aspect, the present invention provides polypeptides as defined and/or disclosed in the present specification.

In an aspect, the present invention provides methods for preparing polypeptides as disclosed in the present specification.

In an aspect, the present invention provides methods of screening as defined and/or disclosed in the present specification.

In an aspect, the present invention provides the use of polypeptides, nucleotide sequences, vectors, and cells as defined herein in methods of screening.

The polypeptides, cells and or methods of the invention are useful in and/or as assays for screening agents, in particular agents exerting an activity on TNFRSF receptors.

Further aspects and preferred embodiments of the invention are provided in the detailed description below and in the appended claims.

### BRIEF DESCRIPTION OF THE DRAWINGS

In the figures,

FIG. 1a schematically represents the first step of the cloning strategy for the preparation of a recombinant polypeptide according to a first embodiment of the present invention, in which a fusion gene is formed by fusing DNA encoding the full length human TNFR1 to DNA encoding intracellular (IC) domain of mouse platelet derived growth factor receptor (PDGFR), a RTK, thereby creating a fusion gene.

FIG. 1b schematically represents a further step of the cloning strategy for the preparation of a recombinant polypeptide according to the first embodiment of the present invention. In particular, the fusion gene shown in FIG. 1a, transferred to vector pDON221, is introduced into the vector pcDNA3.1 Hygro GW to yield the expression vector pcDNA3.1 hygro TNFR1-PDGFR.

FIG. 2 shows fluorescence intensity measured in flow cytometry of HEK293T cells transfected with the expression vector pcDNA3.1 hygro TNFR1-PDGFR. Due to binding of a fluorescent specific monoclonal antibody recognizing TNFR1 to the chimeric receptor, cells expressing the chimeric receptor according to the first embodiment of the invention (solid line) exhibit different fluorescence than the control cells (dotted line, staining with an unspecific monoclonal antibody of the same isotype as the specific monoclonal antibody recognizing TNFR1). An isotype matched control that has no specificity to any component of the cells provides some idea of the amount of non-specific binding that one may get with the specific antibody.

FIG. 3a is a dose response curve obtained in an HTS setting using the Ca²⁺-dependent luminescence of Aequorin cells as indicator of activity of the chimeric receptor according to the first embodiment of the invention following administration of increasing administration of TNF. The dose response curve is established on the basis of the integration of the luminescence emitted in 10 minutes following TNF administration.

FIG. 3b is a dose response curve as FIG. 3a, with the difference that the dose response curve is established on the basis of the intensity of the light response in dependence of applied TNF (max-min).

FIG. 4 shows individual traces of luminescent signal over time following administration of different TNF concentrations ranging from 50 ng/ml to 100 pg/ml to the cells containing, on their surface, the chimeric receptor according to the first embodiment of the invention. One trace corresponds to one sample exposed to a specific concentration.

FIG. 5a shows the luminescent signal (AUC) of cells of the first embodiment of the invention exposed to medium, TNF and TNF together with a TNFR1-specific antibody, respectively. The antibody, binding to the extracellular part of TNFR1, blocks TNF mediated signalling.

FIG. 5b is as FIG. 5a, with the difference that in the right column TNF is co-administered with a PDGFR tyrosine kinase inhibitor instead of the TNFR1-specific antibody. The signalling is blocked as in FIG. 5a, this time due to inactivation of the tyrosine kinase activity of the chimeric receptor of the present invention.

FIG. 6 shows dose response curves of cells of the first embodiment of the invention (squares) and cells transfected to express the full length PDGFR (circles) exposed to increasing concentrations of the same inhibitor used in FIG. 5b. The cells of the invention were exposed to TNF, whereas the other cells were exposed to human PDGF-BB.

FIG. 7 is a scatter plot showing the calcium flux or concentration as area under the curve (AUC) of luminescence units for individual samples containing cells of the first embodiment of the invention exposed to medium (on the left) and to the EC80 concentration of TNF (on the right). The indicated figure of 0.59 corresponds to the Z′-factor of the assay, demonstrating the suitability of the assay for HTS.

FIG. 8a shows a dose response curve obtained with cells according to a second embodiment of the invention. Cells were transfected with a nucleotide sequence encoding a chimeric receptor comprising a truncated TNFR1 (extracellular and transmembrane domain) fused to the cytoplasmic, tyrosine kinase domain of a PDGFR. The light signal reflects intracellular Ca²⁺ concentration, but, in contrast to the setting underlying FIGS. 3a and 3b, is established on the basis of Fluo-4 AM, a cell-permeable, fluorescent Ca²⁺ indicator.

FIG. 8b is as FIG. 8a, but obtained with cells according to a third embodiment of the invention. Cells of this embodiment were transfected with a nucleotide sequence encoding a chimeric receptor comprising a truncated (only extracellular domain) TNFR1 fused to the cytoplasmic tyrosine kinase and the transmembrane domain of a PDGFR.

FIG. 9 shows fluorescence intensity measured in flow cytometry of HEK293T cells transfected with the expression vector pcDNA3.1 hygro DR3(fl)-PDGFR, expressing a nucleotide sequence encoding a chimeric polypeptide comprising the full-length DR3 receptor, in accordance with another embodiment of the invention. DR3 is also known as TNFRSF member 25, another member of the TNFRSF. Due to binding of a fluorescent specific monoclonal antibody recognizing DR3 to the chimeric receptor, cells expressing the chimeric receptor according to the first embodiment of the invention (solid line) exhibit different fluorescence than the control cells (dotted line, staining with an unspecific monoclonal antibody of the same isotype as the specific monoclonal antibody recognizing DR3). An isotype matched control that has no specificity to any component of the cells provides some idea of the amount of non-specific binding that one may get with the specific antibody.

FIG. 10 depicts the dose response curve obtained in an HTS setting using the Ca²⁺-dependent luminescence of Aequorin cells as indicator of activity of the chimeric receptor mentioned with respect to FIG. 9 above, following administration of increasing administration of TL1A (also known as Tumor necrosis factor ligand superfamily member 15 or Vascular endothelial growth inhibitor). The dose response curve is established on the basis of the integration of the luminescence emitted in 10 minutes following TL1A administration.

FIG. 11 shows the individual traces of luminescent signal over time following administration of different TL1A concentrations ranging from 1 ng/ml to 2 μg/ml to the cells containing, on their surface, the chimeric receptor described with respect to FIG. 9 above. One trace corresponds to one sample exposed to a specific concentration.

FIG. 12 depicts the dose response curve obtained in an HTS setting using the Ca²⁺-dependent luminescence of Aequorin cells as indicator of activity of the chimeric receptor FAS (extracellular and transmembrane domains) fused to the cytoplasmic tyrosine kinase domain of PDGFR according to a further embodiment of the invention, following administration of increasing concentrations of FAS ligand (FASL). The dose response curve is established on the basis of the integration of the luminescence emitted in 17 minutes following FASL administration.

FIG. 13 is as FIG. 12, with the difference that the chimeric receptor consists of the extracellular domain of FAS, fused to the transmembrane and cytoplasmic tyrosine kinase domains of PDGFR according to a further embodiment of the invention.

FIG. 14 is as FIGS. 12 and 13, with the difference that the chimeric receptor consists of the full length FAS fused to the death domain of TNFR1 and the cytoplasmic tyrosine kinase domain of PDGFR according to a further embodiment of the invention.

FIG. 15 is as FIGS. 12-14, with the differences that the chimeric receptor consists substantially of the extracellular and transmembrane domains of the FAS receptor fused to the cytoplasmic domain of TNFR1 and the cytoplasmic tyrosine kinase domain of PDGFR, according to a further embodiment of the invention, and that the dose response curve is established on the basis of the integration of the luminescence emitted in 22 minutes following FASL administration.

FIG. 16 depicts the dose response curve obtained in an HTS setting using the Ca²⁺-dependent luminescence of Aequorin cells as indicator of activity of the chimeric receptor TNFR2 full length fused to the death domain of TNFR1 and the cytoplasmic tyrosine kinase domain of PDGFR according to a further embodiment of the invention, following administration of increasing concentrations of TNF. The dose response curve is established on the basis of the integration of the luminescence emitted in 10 minutes following TNF administration.

### DETAILED DESCRIPTION OF THE PREFERRED EMBODIMENTS

The present invention provides chimeric and/or fusion polypeptides comprising at least two parts originating from different proteins. The chimeric polypeptide may comprise at least two amino acid sequence parts. In particular, the chimeric polypeptide functions as a chimeric receptor. The chimeric polypeptide may be provided in the form of a protein isolate, but is generally provided in a cell or on the surface of a cell, in particular embedded in a membrane of a cell, preferably in the plasma membrane.

The chimeric polypeptide preferably comprises a first part, which is taken from and/or substantially identical to a receptor A, or at least a part or stretch thereof, said receptor A being preferably as defined below. Preferably, said first part comprises an amino acid sequence part taken from and/or substantially identical to the amino acid sequence of said receptor A, or preferably comprising the extracellular domain of said receptor A.

For the purpose of the present specifications, the expressions “first part”, “second part” and “third part”, and “fourth part” are used. The words “first”, “second”, “third” and “fourth” are, in principle not used to express any kind of priority or relative importance of the various parts, but are generally used to differentiate the various structural elements of the chimeric polypeptide of the invention for purposes of clarity. Instead of “first part”, one could, for example also use the expression “TNFRSF part”, and instead of “second part”, one could use the expression “RTK-tyrosine kinase part”, for example, or other terms reflecting the function and/or origin of the respective sequence parts. One can also omit the wording “first part”, etc, altogether while referring to the corresponding sequence stretch and/or function. With respect to the third part, this part is only necessary as a separate part in case one does not make use of the transmembrane domain of the TNFRSF receptor or of the RTK receptor.

According to an embodiment, said first part comprises an amino acid sequence that is substantially identical to the full-length amino acid sequence of said receptor A. It is particularly surprising that chimeric receptors comprising a full length target receptor (receptor A) and, in addition, an intracellular portion substantially identical to the one of an RTK (protein B) as defined below constitute a functional signal transduction unit. This is surprising, because, without wishing to be bound by theory, the intracellular portion of such target receptors (receptors A) was previously thought to be obstructive to or to even prevent activation of the intracellular portion of an RTK or at least the transduction of RTK-like signals, due to conformational changes affecting said intracellular part of said receptor A. In particular, one could assume that the intracellular portion of said full length receptor A would, upon binding of an active agent and/or ligand, move a tyrosine kinase portion of the RTK to a spatial position or orientation were RTK-like signals are not transduced. The inventors of the present invention are not aware of any instance were a full-length TNFRSF receptor was fused to a cytoplasmic tyrosine kinase domain of an RTK to yield a functional chimeric polypeptide.

The expression “full length”, according to an embodiment, does also but not only encompasses the situation where an amino acid sequence of a given receptor is completely and/or identically used as occurring in nature. This term preferably also encompasses the situations that one or more amino acids are missing or replaced, in particular functionally not or less relevant amino acids. The expression “full length” preferably means that all functional units of a given receptor, such as ligand binding, transmembrane and intracellular domains, such as recruiting domains and the like, are present. According to a preferred embodiment, the term “full length” means in particular that there is an absence of a truncation of one or more substantial continuous sequence portions, such as one or more substantial portions of the cytoplasmic domain. In particular, the expression “full length” is intended to encompass sequences of receptors in which up to 50, preferably up to 45, 40, 35, 30, 25, 20, 15, 10, 9, 8, 7, 6, 5, 4, 3, 2 or 1 continuous amino acid moieties are missing if compared to the native or original receptor A.

Furthermore, the expression “full length” preferably also encompasses situations where an artificial amino acid sequence is provided, encoded or used, which artificial sequence combines portions of related, similar or homologous proteins, for example as present in different species, in a similar manner and/or in the same order of functional entities and/or portions as they are provided in a particular receptor A or protein B as defined herein.

According to another embodiment, said first part does not comprise the full-length amino acid sequence, but comprises a portion, which is taken from and/or substantially identical to a stretch of the amino acid sequence of said receptor A. Preferably, the first part comprises an amino sequence that is taken from and/or substantially identical to at least a major part of the extracellular, ligand-binding portion of said receptor A, an more preferably the complete extracellular, ligand-binding portion of said receptor A.

The expression “a major part” includes, for the purpose of the present specification, the situation where said first part comprises one or more stretches that are identical to one or more stretches found in said receptor A, so that said entire first part preferably may comprise a continuous stretch that has at least 30%, 40%, 50% or more sequence identity or more, as indicated elsewhere in this specification, if aligned with the extracellular, ligand binding portion of said receptor A.

As becomes clear from the above, said first part is preferably defined so as to encompass any possible amino acid sequence stretch taken from and/or substantially identical to an amino acid sequence of said receptor A, with the proviso that it comprises at least the extracellular, ligand-binding portion, but possibly more than that, for example also including partially or totally the transmembrane domain of said receptor A, and/or partially or totally the intracellular portion of said receptor A.

According to an embodiment, said first part has any one or both of the following capacities and/or retains any one or both of the following functions of said original receptor A:

(a) oligomerization, for example di-, tri- and/or polymerization, with the corresponding extracellular domain of the receptor A and/or with the extracellular domain of another chimeric polypeptide according to the invention;

(b) binding of an agent exhibiting an activity, for example of a natural ligand of the receptor A.

The capacity (a) may actually be and preferably is dependent on binding of a ligand as mentioned under (b).

Regarding the capacity (a) of oligomerization as conferred by said first part of said receptor A it is noted that this preferably includes the capacity of pre-ligand, dimer assembly and thus dimerization, although such dimers are supposed not to be signalling (see publication of (Chan, Francis Ka-Ming, Cytokine 2007, 37(2): 101-107)).

Preferably, the capacity (a) of oligomerization as conferred by said first part of said receptor A encompasses the capacity of trimerization, as it is thought that trimerization is seen as a common initiating event in the TNFRSF signaling cascades (see above). Furthermore, according to an embodiment, said capacity (a) of oligomerization may also refer to the capacity or function of assembly of ligand-receptor trimers into higher complexity structures (n-trimers, hexa-, nona-, dodecamers, etc., as specified above).

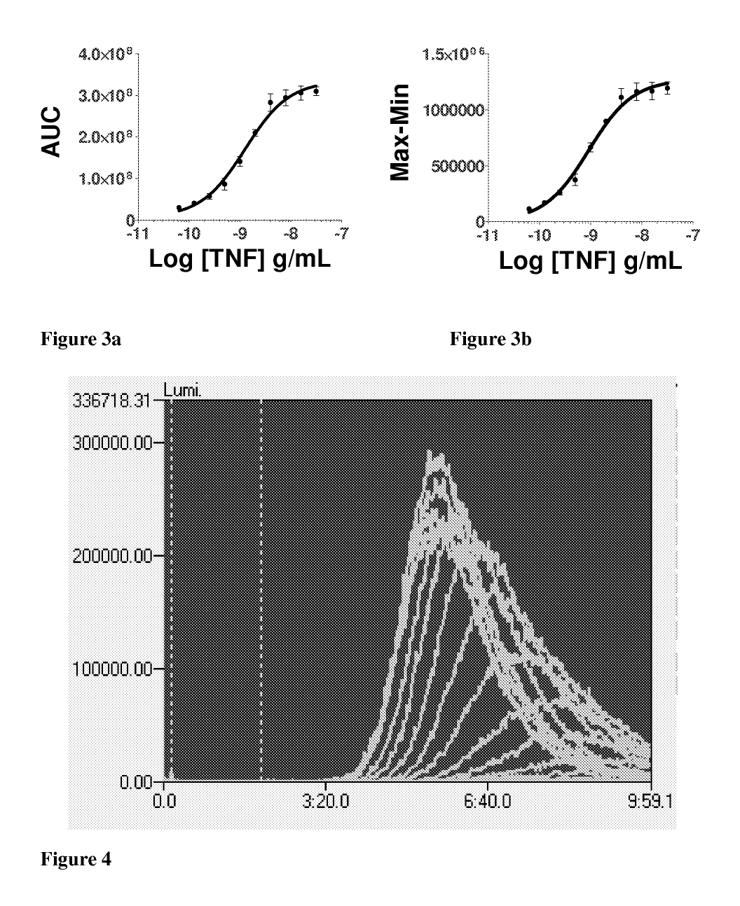

In receptors A, signaling is supposed to be dependent on binding and possibly and/or generally oligomerization, for example dimerization, or even polymerization. Accordingly, the properties or functions (a) and (b) may be determined by the assay as shown in the examples. In particular, said first part may be fused to a second part, wherein said second part is known to be functional, for example because it comprises a functional kinase portion as specifically disclosed in Example 1. If any first part as defined herein, if fused to said second part, is capable of signaling if exposed to its natural ligand as demonstrated in a dose response curve as shown, for example, in FIG. 3a or 3b and the corresponding methodology.

In other words, in said first part, the amino acid sequence taken from and/or substantially identical to the amino acid sequence of said receptor A is sufficiently complete and/or identical to the corresponding portion of said receptor A so as to confer to the chimeric polypeptide of the invention similar and/or preferably substantially the same ligand binding properties, ligand-binding characteristics and/or affinities as the extracellular, ligand binding portion of said original receptor A.

Said receptor A is preferably a receptor selected from receptors of the TNFRSF.

Table 1 below lists exemplary receptors of the TNFRSF and protein accession numbers of receptors in the organisms indicated. Said receptor A, may, for example, be a receptor selected from the receptors listed in Table 1.

TABLE 1
Receptors of the TNFRSF

| TNFRSF | | | | | | |
|---|---|---|---|---|---|---|
| Nomen- | Molecular | | Pan | Canis lupus | | |
| clature | Aliases | Homo sapiens | troglodytes | familiaris | Bos taurus | Mus musculus |
| TNFRSF1A | TNFR type I, | NP_ | XP_ | XP_ | NP_ | NP_ |
| | CD120a, | 001056 | 522334 | 854474 | 777099 | 035739 |
| | TNFAR, | | | | | |
| | p55TNFR, | | | | | |
| | TNFR60 | | | | | |
| TNFRSF1B | TNFR type II, | NP_ | XP_ | XP_ | NP_ | NP_ |
| | CD120b, | 001057 | 514405 | 544562 | 001035580 | 035740 |
| | TNFR80, | | | | | |
| | p75TNFR, | | | | | |
| | TNFBR | | | | | |
| TNFRSF3 | TNFR III, | NP_ | XP_ | XP_ | NP_ | NP_ |
| | LTBR, | 002333 | 508950 | 543855 | 001096698 | 034866 |
| | TNFCR, | | | | | |
| | TNFR-RP, | | | | | |
| | TNFR2-RP | | | | | |
| TNFRSF4 | OX-40, | NP_ | XP_ | XP_ | NP_ | NP_ |
| | ACT35, | 003318 | 513705 | 546720 | 001092513 | 035789 |
| | TXGP1L, | | | | | |
| | CD134 | | | | | |
| TNFRSF5 | CD40, | NP_ | | NP_ | XP_ | NP_ |
| | Bp50, p50 | 001241 | | 001002982 | 581509 | 035741 |
| TNFRSF6 | Fas, CD95, | NP_ | XP_ | XP_ | NP_ | NP_ |
| | APO-1, | 000034 | 001139138 | 543595 | 777087 | 032013 |
| | APT1, | | | | | |
| | TNFRSF6A | | | | | |
| TNFRSF6B | DcR3, TR6, | NP_ | | | NP_ | |
| | M68 | 116563 | | | 001094776 | |
| TNFRSF7 | CD27, | NP_ | XP_ | XP_ | NP_ | NP_ |
| | S152, Tp55, | 001233 | 508952 | 854464 | 001075903 | 001028298 |
| | T14 | | | | | |
| TNFRSF8 | CD30, Ki-1 | NP_ | XP_ | XP_ | XP_ | NP_ |
| | | 001234 | 514397 | 544563 | 871494 | 033427 |
| TNFRSF9 | 4-1BB, | NP_ | XP_ | XP_ | NP_ | NP_ |
| | CDw137, | 001552 | 001157779 | 850336 | 001030413 | 001070977 |
| | ILA | | | | | |
| TNFRSF10A | DR4, | NP_ | XP_ | | XP_ | NP_ |
| | TRAIL-R1, | 003835 | 001158464 | | 001790124 | 064671 |
| | APO-2, | | | | | |
| | CD261 | | | | | |
| TNFRSF10B | DR5, | NP_ | XP_ | | | |
| | TRAIL-R2, | 003833 | 001158136 | | | |
| | KILLER, | | | | | |
| | CD262, | | | | | |
| | TRICK2A, | | | | | |
| | TRICKB | | | | | |
| TNFRSF10C | DcR1, | NP_ | XP_ | | | |
| | TRAIL-R3, | 003832 | 528085 | | | |
| | LIT, TRID, | | | | | |
| | CD263 | | | | | |
| TNFRSF10D | DcR2, | NP_ | XP_ | | | |
| | TRAIL-R4, | 003831 | 528087 | | | |
| | TRUNDD, | | | | | |
| | CD264 | | | | | |
| TNFRSF11A | RANK, | NP_ | XP_ | | | |
| | ODFR, | 003831 | 528087 | | | |
| | TRANCE-R, | | | | | |
| | CD265 | | | | | |
| TNFRSF11B | OPG, TR1, | NP_ | XP_ | XP_ | NP_ | NP_ |
| | OCIF | 002537 | 519921 | 539146 | 001091525 | 032790 |
| TNFRSF12A | TWEAK-R, | NP_ | XP_ | | XP_ | NP_ |
| | Fn14, FGF- | 057723 | 001165479 | | 874792 | 038777 |
| | inducible | | | | | |
| | 14, CD266 | | | | | |
| TNFRSF13B | TACI, | NP_ | XP_ | XP_ | XP_ | NP_ |
| | CD267 | 036584 | 001161317 | 851957 | 875375 | 067324 |
| TNFRSF13C | BAFF-R, | NP_ | XP_ | XP_ | XP_ | NP_ |
| | CD268, | 443177 | 001154286 | 849061 | 875941 | 082351 |
| | BR3 | | | | | |
| TNFRSF14 | HVEM, | NP_ | XP_ | XP_ | XP_ | NP_ |
| | TR2, | 003811 | 513730 | 549666 | 875941 | 082351 |
| | LIGHT-R, | | | | | |
| | ATAR, | | | | | |
| | HVEA | | | | | |
| TNFRSF16 | NGF-R, | NP_ | XP_ | XP_ | XP_ | NP_ |
| | NTR, | 443177 | 001154286 | 849061 | 875941 | 082351 |
| | p75NGFR, | | | | | |
| | CD271 | | | | | |
| TNFRSF17 | BCMA, | NP_ | XP_ | | | NP_ |
| | BCM, | 001183 | 523298 | | | 035738 |
| | TNFRSF13, | | | | | |
| | TNFRSF13a, | | | | | |
| | CD269 | | | | | |
| TNFRSF18 | AITR, | NP_ | XP_ | XP_ | XP_ | NP_ |
| | GITR | 004186 | 001144452 | 848560 | 594408 | 033426 |
| TNFRSF19 | TROY, | NP_ | XP_ | XP_ | | NP_ |
| | TAJ, TAJ-α, | 061117 | 001151665 | 543168 | | 038897 |
| | TRADE | | | | | |
| TNFRSF19L | RELT | NP_ | XP_ | XP_ | XP_ | NP_ |
| | | 689408 | 001174800 | 542318 | 582052 | 796047 |
| TNFRSF21 | DR6, Death | NP_ | XP_ | XP_ | NP_ | NP_ |
| | receptor 6 | 055267 | 001145645 | 852414 | 001070379 | 848704 |
| TNFRSF22 | SOBa; | NP_ | | | | |
| | Tnfrh2, | 076169 | | | | |
| | Tnfrsf1al2, | | | | | |
| | mDcTrailr2 | | | | | |
| TNFRSF23 | mSOB, | | | | | NP_ |
| | Tnfrh1, | | | | | 076169 |
| | mDcTrailr1 | | | | | |
| TNFRSF25 | DR3, APO-3, | NP_ | XP_ | XP_ | XP_ | NP_ |
| | TRAMP, | 683866 | 001165991 | 546752 | 001252043 | 149031 |
| | TRS, WSL-1, | | | | | |
| | LARD, | | | | | |
| | DDR3, | | | | | |
| | WSL-LR | | | | | |

Preferably, receptor A is a receptor selected from type 1 (extracellular N terminus) receptors of the TNFRSF. Currently there are 29 TNFRSF members. Preferably, receptor A is selected from TNFRs, and most preferably from TNFR1 and TNFR2.

It is particularly surprising that the chimeric polypeptide comprising the extracellular domain of a TNFRSF receptor is suitable for the purposes of the present invention. In vivo, receptors of the TNFRSF are believed to exist in a pre-ligand, dimer assembly (Chan, Francis Ka-Ming, Cytokine 2007, 37(2): 101-107). Pre-ligand dimerization is, however, expected to activate the cytoplasmic tyrosine kinase domain of said chimeric polypeptides and to induce RTK-like signals, since RTKs are active as dimers. Surprisingly, however, no RTK signal is measured in the absence of a ligand of the chimeric polypeptide and/or receptor A.

The first amino acid sequence part of said chimeric polypeptide of the invention preferably comprises and more preferably consists of an amino acid sequence taken from and/or substantially identical to the amino acid sequence of said receptor A, or at least the extracellular, ligand binding part thereof. Similar terminology is used with respect to receptor B, discussed in more detail further below.

The expression “substantially identical to” for the purpose of the present invention and in particular with respect to the first part of the chimeric polypeptide, refers to amino acid sequences having at least 50%, preferably at least 55%, 60%, 65%, 70%, 75%, 80%, 82%, 85%, 87%, 90%, 91%, 92%, 93%, 94%, 95%, 96%, 97%, 98%, 99% sequence identity with the corresponding sequence or sequence portion or stretch (for example, the extracellular portion) of receptor A, for example.

For the purpose of the present specification, sequence identity percentage is determined by using the basic protein blast on the internet (http://blast.ncbi.nlm.nih.gov) with preset standard parameters and database selections. This sequence comparison tool is based on algorithms detailed in the two following publications: Stephen F. Altschul, Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. Stephen F. Altschul, John C. Wootton, E. Michael Gertz, Richa Agarwala, Aleksandr Morgulis, Alejandro A. Schäffer, and Yi-Kuo Yu (2005) “Protein database searches using compositionally adjusted substitution matrices”, FEBS J. 272:5101-5109.

Standard parameters include the selection of blastp (protein-protein BLAST, automatic adjustment of parameters to short input sequences; expect threshold 10, word size 3, use of the matrix BLOSUM62; Gap costs: existence: 11, extension 1; conditional compositional score matrix adjustment, no filters and no masking).

Sequence identity of a sequence of comparison with respect to an original sequence is reduced when, for example, any one of the compared or the original sequence lacks amino acid residues, has additional amino acid residues and/or has one or more amino acid residue substituted by another residue. Sequences having as little as 50% sequence identity with any sequence as defined herein may still provide functional, that is, having, independently, ligand binding functionality, tyrosine kinase functionality, transmembrane functionality, and possibly further and/or other functionalities as defined herein, and are thus suitable to meet the objectives of the invention.

In the case of the extracellular, ligand-binding portion of said first part of said chimeric polypeptide, taken from and/or substantially identical to said receptor A, generally higher sequence identity percentages if compared to receptor A are preferred, in order to retain to a large extent the ligand binding and/or oligomerization properties of the original receptor A. According to a preferred embodiment, for this portion of the first part, there is at least 80% and more (as indicated above) sequence identity with receptor A. With respect to transmembrane portions and/or the intracellular portion taken of RTKs (receptor B, discussed below), lower sequence identity levels may be sufficient to maintain the function of the chimeric polypeptide of the invention.

According to an embodiment, “substantially identical” refers to sequence identities of at least 80% and 60% identity of said first and second parts with said amino acid sequence portion of said receptors A and B, respectively, more preferably at least 85% and 70%, most preferably at least 90% and 80%. However, sequence identities of said first and second part may be independently selected, preferably in dependence of the functionalities as described elsewhere in this specification.

The chimeric polypeptide comprises a second part, which is taken from and/or substantially identical to an intracellular, signaling kinase portion of a receptor B, said receptor B being selected from receptor tyrosine kinases (RTKs). Preferably, said second part is an amino acid sequence part taken from and/or substantially identical to the amino acid sequence of an intracellular, signaling kinase portion of a receptor B. The expression “substantially identical” has, independently, the meaning as detailed above.

According to an embodiment, the second part comprises the entire intracellular portion of said receptor B.

Preferably, said receptor B is preferably selected from receptors of the RTK super family (RTKSF). More preferably, receptor B is selected from RTKs, which are not present in a disulfide bridged dimer in the non-active state. RTKs of this latter type, such as the insulin receptor, are activated by a mode of activation that is different from ligand-induced dimerization. Preferably, the said receptor B is selected from RTKs that are characterised by ligand-induced dimerization.

RTKs represent classical examples of surface receptors whose activation relies upon dimerization and/or ligand-induced global conformational changes. RTK are single-pass membrane proteins with an extracellular ligand-binding domain and an intracellular kinase domain. Members of this large group of membrane proteins have been classified on the basis of their structural and ligand affinity properties (Fantl et al. 1993 Annu. Rev. Biochem. 62, 453). The RTK family includes several subfamilies, including the epidermal growth factor receptors (EGFRs or ErbBs), the fibroblast growth factor receptors (FGFRs), the insulin and the insulin-like growth factor receptors (IR and IGFR), the platelet derived growth factor receptors (PDGFRs), the vascular endothelial growth factor receptors (VEGFRs), the hepatocyte growth factor receptors (HGFRs), and the nerve growth factor receptors (NGFRs) (van der Geer et al. 1994 Annu. Rev. Cell Biol. 10, 251). The receptor B may be selected from any one of the aforementioned RTKs. According to a preferred embodiment, receptor B is selected from PDGFRs, EGFRs, FGFRs, and VEGFRs. To mention a few specific examples, mouse PDGFR is available under accession number NM_{—}008809.1 human EGFR is available under accession number NM_{—}005228, human FGFR is available under accession number NM_{—}015850.3, human VEGFR is available under accession number NM_{—}002019.

Table 2 below lists receptors of the RTK super family (RTKSF). Said receptor B may, for example, be selected from the receptors listed in Table 2 below.

TABLE 2
Receptors of the RTK super family (RTKSF)

| Nomen- | Molecular | | Pan | Canis lupus | | |
|---|---|---|---|---|---|---|
| clature | Aliases | Homo sapiens | troglodytes | familiaris | Bos taurus | Mus musculus |
| ALK | Kil | NP_ | | XP_ | XP_ | NP_ |
| | | 004295 | | 540136 | 616782 | 031465 |
| LTK | TYK1 | NP_ | XP_ | | | NP_ |
| | | 002335 | 001149706 | | | 976220 |
| AXL | UFO, | NP_ | | XP_ | XP_ | NP_ |
| | Tyro7, | 001690 | | 541604 | 594754 | 033491 |
| | Ark | | | | | |
| MER | MERTK, | NP_ | XP_ | XP_ | XP_ | NP_ |
| | NYK, | 006334 | 515690 | 540175 | 580552 | 032613 |
| | Eyk | | | | | |
| TYRO3 | RSE, | NP_ | | XP_ | XP_ | NP_ |
| | SKY, | 006284 | | 544633 | 001253887 | 062265 |
| | BRT, | | | | | |
| | DTK, | | | | | |
| DDRI | CAK, | NP_ | XP_ | XP_ | | NP_ |
| | TRKE, | 054699 | 001150123 | 532062 | | 031610 |
| | NEP, | | | | | |
| | NTRK4, | | | | | |
| DDR2 | TKT, | NP_ | XP_ | XP_ | NP_ | NP_ |
| | TYRO10, | 001014796 | 513955 | 536144 | 001077189 | 072075 |
| | NTRKR | | | | | |
| EGFR | ERBB, | NP_ | XP_ | XP_ | XP_ | NP_ |
| | ERBB1 | 005219 | 001156495 | 533073 | 592211 | 997538 |
| ERBB2 | HER2, | NP_ | | NP_ | | |
| | Neu, | 004439 | | 001003217 | | |
| | NGL | | | | | |
| ERBB3 | HER3 | NP_ | XP_ | XP_ | NP_ | NP_ |
| | | 001973 | 509131 | 538226 | 001096575 | 034283 |
| ERBB4 | HER4 | NP_ | XP_ | XP_ | | XP_ |
| | | 005226 | 516067 | 545629 | | 136682.7 |
| EPHA1 | EPH, | NP_ | XP_ | XP_ | XP_ | NP_ |
| | EPHT | 005223 | 519451 | 539851 | 604305 | 076069 |
| EPHA2 | ECK, | NP_ | XP_ | XP_ | XP_ | NP_ |
| | Sek2, | 004422 | 513064 | 864941 | 590380 | 034269 |
| | Myk2 | | | | | |
| EPHA3 | HEK, | NP_ | XP_ | XP_ | XP_ | NP_ |
| | ETK1, | 005224 | 001136396 | 545052 | 618140 | 034270 |
| | Tyro4, | | | | | |
| | Mek4, Ce | | | | | |
| EPHA4 | HEK8, | NP_ | XP_ | XP_ | | NP_ |
| | Tyro1, | 004429 | 001164795 | 536084 | | 031962 |
| | Sek1, | | | | | |
| | Cek8 | | | | | |
| EPHA5 | HEK7, | NP_ | XP_ | | | NP_ |
| | Ehk1, | 004430 | 001164976 | | | 031963 |
| | Bsk, | | | | | |
| | Cek7 | | | | | |
| EPHA6 | DKFZp4 | NP_ | XP_ | XP_ | XP_ | NP_ |
| | 34C1418, | 001073917 | 516608 | 849887 | 001788053 | 031964 |
| | Ehk2 | | | | | |
| EPHA7 | HEK11, | NP_ | | XP_ | XP_ | NP_ |
| | Mdk1, | 004431 | | 853923 | 611161 | 034271 |
| | Ebk, | | | | | |
| | Ehk3, | | | | | |
| EPHA8 | HEK3, | NP_ | | XP_ | XP_ | NP_ |
| | KIAA1459, | 065387 | | 544509 | 599537 | 031965 |
| | Eek, | | | | | |
| | Cek10 | | | | | |
| EPHB1 | NET, | NP_ | XP_ | XP_ | XP_ | NP_ |
| | EPHT2, | 004432 | 001150963 | 542791 | 614602 | 775623 |
| | HEK6, | | | | | |
| | Elk, | | | | | |
| EPHB2 | HEK5, | NP_ | XP_ | XP_ | XP_ | NP_ |
| | ERK, | 004433 | 513189 | 544506 | 885612 | 034272 |
| | DRT, | | | | | |
| | EPHT3, | | | | | |
| EPHB3 | HEK2, | NP_ | XP_ | XP_ | XP_ | NP_ |
| | Tyro6, | 004434 | 516918 | 545232 | 613645 | 034273 |
| | Mdk5, | | | | | |
| | Sek4 | | | | | |
| EPHB4 | HTK, | NP_ | XP_ | XP_ | XP_ | NP_ |
| | Tyro11, | 004435 | 519269 | 546948 | 874493 | 034274 |
| | Mdk2, | | | | | |
| | Myk1 | | | | | |
| EPHB6 | HEP, | NP_ | XP_ | XP_ | | NP_ |
| | Mep, | 004436 | 519443 | 532743 | | 031706 |
| | Cek1 | | | | | |
| FGFR1 | FLT2, | NP_ | XP_ | XP_ | NP_ | NP_ |
| | bFGFR, | 056934 | 519715 | 856878 | 001103677 | 034336 |
| | FLG, | | | | | |
| | N-SAM | | | | | |
| FGFR2 | KGFR, | NP_ | XP_ | NP_ | XP_ | NP_ |
| | K-SAM, | 000132 | 001157227 | 001003336 | 001789758 | 034337 |
| | Bek, | | | | | |
| | CFD1, J | | | | | |
| FGFR3 | HBGFR, | NP_ | | XP_ | NP_ | NP_ |
| | ACH, | 000133 | | 545926 | 776743 | 032036 |
| | Cek2 | | | | | |
| FGFR4 | | NP_ | XP_ | XP_ | XP_ | NP_ |
| | | 998812 | 518127 | 546211 | 602166 | 032037 |
| IGF1R | JTK13 | NP_ | XP_ | XP_ | XP_ | NP_ |
| | | 000866 | 001136377 | 858671 | 606794 | 034643 |
| INSR | IR | NP_ | | XP_ | XP_ | NP_ |
| | | 000199 | | 542108 | 590552 | 034698 |
| INSRR | IRR | NP_ | | XP_ | XP_ | NP_ |
| | | 055030 | | 547526 | 001254386 | 035962 |
| MET | HGFR | NP_ | XP_ | NP_ | NP_ | NP_ |
| | | 001120972 | 001138791 | 001002963 | 001013017 | 032617 |
| RON | MST1R, | NP_ | XP_ | XP_ | XP_ | NP_ |
| | CDw136, | 002438 | 001166551 | 533823 | 603857 | 033100 |
| | Fv2, | | | | | |
| | STK, | | | | | |
| MUSK | Nsk2, | NP_ | XP_ | XP_ | XP_ | NP_ |
| | Mlk1, | 005583 | 001146498 | 538784 | 591182 | 001032205 |
| | Mlk2 | | | | | |
| CSF1R | FMS, | NP_ | | XP_ | NP_ | NP_ |
| | C-FMS, | 005202 | | 546306 | 001068871 | 001032948 |
| | CD115 | | | | | |
| Flt3 | FLK2, | NP_ | XP_ | NP_ | XP_ | NP_ |
| | STK1, | 0041110 | 509601 | 001018647 | 590263 | 034359 |
| | CD135 | | | | | |
| Kit | Sfr, | NP_ | XP_ | NP_ | XP_ | NP_ |
| | CKIT | 000213 | 517285 | 001003181 | 612028 | 066922 |
| PDGFRA | | NP_ | | XP_ | XP_ | NP_ |
| | | 006197 | | 532374 | 590921 | 001076785 |
| PDGFRB | PDGFR, | NP_ | XP_ | NP_ | XP_ | NP_ |
| | JTK12 | 002600 | 518034 | 001003382 | 001790034 | 032835 |
| PTK7 | CCK4, | NP_ | XP_ | XP_ | XP_ | NP_ |
| | KLG | 002812 | 518486 | 538929 | 869603 | 780377 |
| RET | MEN2A/B, | NP_ | | XP_ | | NP_ |
| | HSCR1, | 066124 | | 543915 | | 033076 |
| | MTC1 | | | | | |
| ROR1 | NTRKR1 | NP_ | XP_ | XP_ | XP_ | NP_ |
| | | 005003 | 513458 | 546677 | 001789312 | 038873 |
| ROR2 | NTRKR2 | NP_ | XP_ | XP_ | | NP_ |
| | | 004551 | 520126 | 541309 | | 038874 |
| ROS1 | MCF3 | NP_ | XP_ | XP_ | | NP_ |
| | | 002935 | 527487 | 541215 | | 035412 |
| RYK | Vik, Mrk | NP_ | | XP_ | XP_ | NP_ |
| | | 002949 | | 534269 | 001249767 | 038677 |
| TEK | TIE2 | NP_ | XP_ | | NP_ | NP_ |
| | | 000450 | 520519 | | 776389 | 038718 |
| TIE | TIE1, | NP_ | XP_ | XP_ | NP_ | NP_ |
| | JTK14 | 005415 | 001173341 | 539652 | 776390 | 035717 |
| NTRK1 | TRK, | NP_ | XP_ | XP_ | XP_ | XP_ |
| | TRKA | 002520 | 001145942 | 547525 | 613650 | 283871 |
| NTRK2 | TRKB | NP_ | XP_ | XP_ | NP_ | NP_ |
| | | 001018074 | 001135401 | 856422 | 001068693 | 001020245 |
| NTRK3 | TRKC | NP_ | NP_ | XP_ | XP_ | NP_ |
| | | 001012338 | 001029295 | 851384 | 585006 | 032772 |
| VEGFR1 | FLT1 | NP_ | XP_ | XP_ | XP_ | NP_ |
| | | 002010 | 509605 | 534520 | 001249769 | 034358 |
| VEGFR2 | KDR, | NP_ | XP_ | XP_ | NP_ | NP_ |
| | FLK1 | 002244 | 517284 | 539273 | 001103740 | 034742 |
| VEGFR3 | FLT4, | NP_ | XP_ | XP_ | XP_ | NP_ |
| | PCL | 891555 | 518160 | 538585 | 001789701 | 032055 |
| AATYK | AATK, | | | | | |
| | KIAA0641 | NP_ | | | XP_ | NP_ |
| | | 001073867 | | | 588863 | 031403 |
| AATYK2 | KIAA1079, | NP_ | XP_ | XP_ | | NP_ |
| | BREK, | 055731 | 001134909 | 851196 | | 001074578 |
| | cprk, | | | | | |
| AATYK3 | KIAA1883; | NP_ | | | XP_ | NP_ |
| | LMR3; | 001073903 | | | 001789580 | 001005511 |
| | TYKLM | | | | | |

According to an embodiment, The expression “substantially identical to” for the purpose of the present invention and in particular with respect to the second part of the chimeric polypeptide, refers to amino acid sequences having at least 50%, preferably at least 55%, 60%, 65%, 70%, 75%, 80%, 82%, 85%, 87%, 90%, 91%, 92%, 93%, 94%, 95%, 96%, 97%, 98%, 99% sequence identity with the corresponding sequence portion or stretch of receptor B, for example.

According to an embodiment, said second part has any one or both of the following capacities and/or retains any one or both of the following functions of said receptor B:

(c) oligomerization, in particular dimerization, with the corresponding intracellular domain of the receptor B and/or with the intracellular portion of another chimeric polypeptide according to the invention;

(d) tyrosine kinase activity.

As becomes clear from the discussion above and elsewhere in this specification, the capacity or function of oligomerization of said receptor B that may preferably be retained by the second part of the chimeric polypeptide may not necessarily be or result in the same type of oligomerization as of said first part/receptor A. In particular, in the case of the second part, the term oligomerization preferably exclusively refers to dimerization.

Without wishing to be bound by theory, it is also supposed that the function or capacity of oligomerization of said second part may encompass or even consist substantially of a type of trans-oligomerization with a corresponding part of another individual chimeric polypeptide. Therefore, without wishing to be bound by theory it is speculated that tyrosine kinase activity of the chimeric polypeptide of the invention can occur as a result of di- or oligomerization of oligomerized chimeric polypeptides. In other words, it is possible that an oligomeric receptor complex formed by the oligomerization of two (or three, etc.) first parts of two (or three, etc.) chimeric polypeptides following ligand binding, needs subsequently oligomerizing with a corresponding oligomeric receptor complex.

In an analogous manner to the indications above with respect to receptor A, the properties (c) and/or (d) of the second part may be determined on the basis of the methodology as shown in the examples. If a given second part, if combined with one of the functional first parts as disclosed in the examples results in a chimeric polypeptide capable of tyrosine kinase mediated signalling, said properties (c) and (d) are most probably achieved by said second part.

In said second part, the amino acid sequence taken from and/or substantially identical to the amino acid sequence of the intracellular, signaling kinase portion of a receptor B is preferably sufficiently complete and/or identical to the respective portion of said receptor B so as to confer to the chimeric polypeptide of the invention similar and/or preferably substantially identical RTK characteristics, such as one or more selected from the generation of an RTK-like signal, tyrosine kinase activity, in particular tyrosine kinase auto- and/or transphosphorylation activity, and oligomerization with an intracellular domain of an RTK. Without wishing to be bound by theory, it is believed that the intracellular kinase portion, in order to transduce a signal, needs to be capable of trans- and/or autophosphorylation. This means that two kinase portions are in a relationship wherein the one cytoplasmic tyrosine kinase domain phosphorylates the other and vice versa, and each one possibly phosphorylates tyrosine residues of itself. Tyrosine autophosphorylation is then believed to recruit and activate a variety of signaling proteins.

The intracellular domain of RTKs generally comprises the tyrosine kinase domain and additional regulatory sequences that are subjected to autophosphorylation and phosphorylation by heterologous protein kinases. According to an embodiment, said second part comprises an amino acid sequence taken from and/or substantially identical to the tyrosine kinase domain and also the additional regulatory sequences. Preferably, the second part comprises at least the regulatory sequences necessary for the generation of an RTK-like signal.

The chimeric polypeptide of the invention comprises, for example in the form of a third part, a transmembrane domain situated between the extracellular, ligand-binding portion of said receptor A and the intracellular, kinase portion of said receptor B. The transmembrane domain preferably connects and/or links said first and second parts together. In this way, a chimeric transmembrane receptor is formed.

In principle, the transmembrane domain may be of any structure, and may thus be selected from transmembrane domains comprising one or a stable complex of several alpha helices, a beta barrel, a beta helix and any other structure. According to a preferred embodiment, the transmembrane is a single alpha helix.

Conveniently, the transmembrane domain stems from any one of the two receptors, receptor A or receptor B. Accordingly, if the first part of the chimeric protein comprises an amino acid sequence taken from and/or substantially identical to the full-length amino acid sequence of receptor A, a transmembrane domain is already present in the chimeric polypeptide. The same is true if the first part comprises substantially the extracellular domain and the transmembrane domain of said receptor A but not its intracellular part (truncated receptor A). On the other hand, the present invention encompasses the possibility that said first part comprises only the extracellular, ligand binding part of said receptor A (also truncated). In this case, the transmembrane domain may be selected from any other transmembrane domain. Conveniently, the transmembrane domain of the receptor B may be used, for example. In this case, the second part of the chimeric polypeptide of the invention comprises, for example, the amino acid sequence taken from and/or substantially identical to the amino acid sequence stretching in a continuous manner from the N-terminus of the transmembrane to the C-terminus of the intracellular RTK domain.

As the skilled person will understand, the origin of the transmembrane portion is generally not relevant, but it is particularly convenient in terms of construct preparation if the chimeric polypeptide contains a transmembrane domain of one of the two mandatory parts of the chimeric polypeptide (TNFRSF receptor or RTK receptor) at the appropriate position. This is, of course, because these receptors are themselves transmembrane receptors that possess a transmembrane domain. It is thus particularly convenient to use at least the extracellular and the transmembrane domains of the receptor A. Accordingly, the C-terminus end of the truncated receptor A is fused to the N-terminus of the intracellular domain of the truncated receptor B (with or without the intracellular, cytoplasmic domain of receptor A). Alternatively, the transmembrane portion of receptor B is used. Accordingly, the N-terminus of the truncated receptor B is fused to the C-terminus of the extracellular portion of truncated receptor A. The present invention does not exclude the possibility that the chimeric polypeptide comprises part of the transmembrane domain of a receptor A and part of the transmembrane domain of a receptor B, fused in such a way so as to form a “chimeric transmembrane domain”.

According to an embodiment, the part comprising the transmembrane domain (for example, the third part) has at least 50%, preferably at least 55%, 60%, 65%, 70%, 75%, 80%, 82%, 85%, 87%, 90%, 91%, 92%, 93%, 94%, 95%, 96%, 97%, 98%, 99% sequence identity with the transmembrane portion of any one selected from receptors A and receptors B. In more general terms, the transmembrane domain may be taken from or be substantially identical to the transmembrane domain of any type 1 single pass transmembrane receptor (e.g. cytokine receptors, receptors of the TGFβ super family, interleukin receptors), or even other transmembrane receptors. Preferably, the transmembrane domain is an a-helical single pass transmembrane domain.

The transmembrane domain preferably provides the function of anchoring the chimeric polypeptide in a membrane of cells harbouring the chimeric polypeptide, for example cells expressing a nucleotide sequence encoding the chimeric polypeptide. Preferably, the transmembrane domain is suitable to keep and/or stabilise the chimeric polypeptide in the plasma membrane of the cells.

According to an embodiment, the polypeptide of the invention comprises one or more death domain(s). The death domain may be included in part 1, for example, or in any other part. It is preferably located between the transmembrane domain and the cytoplasmic portion of receptor B (for example, part 2). The death domain may be the death domain possibly contained in said selected receptor A. Alternatively, the death domain may be from a different receptor, and may thus be independently be selected (see examples below). The invention thus encompasses that the chimeric polypeptide comprises amino acid sequence parts taken from three different receptors. In particular, the polypeptide may comprise a sequence part comprising an amino acid sequence taken from and/or substantially identical to a death domain. This part may be considered a fourth part, in particular if not contained in said first part, or possibly in said second or third part. The function and characteristics of death domains has been reported in the literature. Death domains form an own protein domain super family, which is designated with accession number c102420 and PSSM ID number 141404 at the CNBI conserved domains database. In particular, conserved domains pfam00531 and smart00005 are conserved domains of the death superfamily.

A death domain of a sequence will generally be recognized when the sequence is entered at the conserved domain search mask (http://www.ncbi.nlm.nih.gov/Structure/cdd/wrpsb.cgi), using the defaults settings (allowing for the low-complexity filter in the concise result mode), with the exception of the expect value (E-value) threshold, which may be set to 1.0, preferably 0.1, and most preferably to the default value of 0.01. For literature see: Marchler-Bauer A et al. (2009), “CDD: specific functional annotation with the Conserved Domain Database.”, Nucleic Acids Res.37(D)205-10.

The presence of domains, such as extracellular, transmembrane and cytoplasmic domains, or substantially full-length sequences of receptors of the TNFRSF and/or of receptor tyrosine kinases, including the domains of embodiments of preferred receptors as defined herein and/or domains thereof may also be determined using this method.

According to this method, position-specific scoring matrices (PSSMs) derived from input “reference” sequences are used to identify conserved domains, such as the death domain, using RPS-BLAST (Reverse Position-Specific BLAST).

In the conserved domain database, a consensus sequence (most frequently occurring residue at each position) of the conserved domain is established, and, in sequence comparisons, alignment of a query sequence with the consensus sequence is shown. The consensus sequence of the pfam00531 death domain is:

(PSSM ID.: 109582)
DKLCALLDELLGKDWRELARKLGLSESEIDEIEQENPGLRSPTYELLR
LWEQRHGENATVGELLEALRKLGRRDAAELIESIL.

Specifically, conserved amino acid moieties in the consensus sequence are Gly12, Trp15, Leu18, Ala19, Arg20, Leu22, Gly23, Ile29, Ile32, Glu33, Pro37, Ser41, Pro42, Tyr44, Leu46, Leu47, Trp50, Gln52, Arg53, His54, Gly55, Ala58, Thr59, Leu63, Ala66, Leu67, Gly71, Arg72, Asp74, Glu77, and Ile79 (underlined above). These amino acids at these positions have a score of at least 5, at least 6 or higher. According to an embodiment, a death domain in accordance with the present invention is a sequence, when aligned with the consensus sequence as indicated above, can be aligned with and comprises at least 2, 3, 4, 5, 6, 7, 8, 9, and most preferably at least 10 identical amino acids of the above list of particularly conserved amino acids. Most preferably, and possibly in addition to the above criterion, a death domain in a sequence is present, if, when aligned with the consensus sequence, conserves one, a selection of two and preferably all three of Trp15, Ile29, and Trp50 of the consensus sequence. Trp50 is the most conserved amino acid, appearing in more than 80% of all sequences found to have a death domain.

As an example, the sequence of human TNFR1 used in for the purpose of the present invention (SEQ. ID. NO.: 2, aa1-455), comprises a death domain (aa359-438), and has the following amino acid moieties in common that can be aligned with the pfam00531 consensus sequence: Leu3(359), Ala5(361), Trp15(371), Glu17(373), Arg20(376), Leu22(378), Gly23(379), Leu24(380), Ser25(381), Glu28(384), Ile29(385), Asp30(386), Glu33(389), Asn36(392), Leu39(396), Arg40(397), Tyr44(401), Leu47(404), Trp50(407), Arg53(410), Ala58(416), Thr59(417), Leu63(421), Leu67(425), Arg68(426), Glu77(435), Ile79(437), Glu80(438). Accordingly, the death domain of hTNFR1 has 16 identical amino acids that can be brought in alignment with the above consensus sequence of the pfam00531 conserved domain.

Further or other death domains can be aligned with conserved domain smart 0005. The above criteria may be independently used to determine the presence of a death domain by examining the presence of specifically conserved amino acid moieties with a score of at least 5 or at least 6 present in a query sequence.

According to an embodiment, the chimeric polypeptide comprises a sequence stretch that has at least 50%, preferably at least 55%, 60%, 65%, 70%, 75%, 80%, 82%, 85%, 87%, 90%, 91%, 92%, 93%, 94%, 95%, 96%, 97%, 98%, 99% sequence identity with the death domain of any one of receptor A, in as far as applicable, for example TNFR1, and/or in particular with the consensus sequence of the pfam00531 death domain indicated above.

The death domain, if present, is a complete, that is, functional death domain, which is capable of undergoing conformational change, for example conformational re-orientation and/or unfolding of the stem cc-helix (helices 5 and 6), following ligand binding. Without wishing to be bound by theory, the present inventors believe that the presence of a death domain may assist in the preferential orientation of the RTK domain within the cell plasma so as to be able to be activated and to transduce a signal.

According to an embodiment, the chimeric polypeptide comprises a death domain that is taken from and/or substantially identical to the death domain of the TNFR1. According to an embodiment, this applies in particular if the chimeric polypeptide comprises a full length amino acid sequence of a receptor A or, besides the extracellular portion of a receptor A, the intracellular portion of a receptor A, for example another receptor A. In this regard, according to an embodiment, a functional polypeptide that was prepared in the examples comprises the extracellular portion of a first receptor A (e.g. FAS) and the cytoplasmic portion of a second receptor A (e.g. TNFR1), besides said second part. According to this embodiment, the chimeric polypeptide comprises a death domain of TNFR1.

According to another embodiment, a functional polypeptide that was prepared in the examples comprises substantially the full length amino acid sequence of a first receptor A (e.g. TNFR2) and the death domain of TNFR1, besides said second (RTK-) part.

According to an embodiment, the chimeric polypeptide lacks a cytoplasmic portion of a receptor A, or, in case the chimeric polypeptide comprises a cytoplasmic portion of a receptor A, said chimeric polypeptide preferably comprises a death domain, in particular the death domain of TNFR1. This applies in particular if said cytoplasmic portion of a receptor A is provided on the N-terminal side of the second part of said chimeric polypeptide.

It is found that the present invention does also work if a death domain is absent. In this case, however, it is preferable that the RTK domain is situated close to the plasma membrane. Preferably, in the chimeric polypeptide of the invention, the RTK domain follows immediately the transmembrane domain, or is separated by a relatively short linker, spacer or other amino acid sequence to the RTK domain. Preferably, between the gap between the last amino acid moiety of the transmembrane domain at the inner side of the plasma membrane and the first amino acid of the following RTK domain spans 80 or less, preferably 70, 50, 40, 30, 20, 10, 5 or less amino acid moieties.

Below, constitutions in terms of amino acid sequences and/or amino acid sequence domains, portions or parts comprised in different embodiments of chimeric polypeptides encompassed by the present invention are schematically shown.

1. TNFRSF (full length)—RTK (intracellular domain);

2. TNFRSF (extracellular and transmembrane domains)—RTK (intracellular domain);

3. TNFRSF (extracellular domain)—RTK (transmembrane and intracellular domains);

4. TNFRSF (extracellular domain)—transmembrane domain (any origin)—RTK (intracellular domain);

5. TNFRSF (full-length) (but not TNFR1)—death domain—RTK (intracellular domain); 6. TNFRSF (extracellular and transmembrane domains) (but not TNFR1)—death domain of TNFR1—RTK (intracellular domain).

Amino acid moieties or sequences having or, independently, not having further functionalities, may or may not, independently, be provided terminally and in positions indicated with “-”.

According to an embodiment, the encoded TNFRSF domains and the encoded RTK as shown, for example, under no. 1-6 above, are linked (for example, functionally or structurally linked or joined), for example translationally linked, for example as a fusion protein.

According to an embodiment, the chimeric polypeptide of the invention is a chimeric transmembrane protein, preferably a chimeric transmembrane receptor. Preferably, the chimeric polypeptide has an extracellular N-terminus and an intracellular C-terminus. In the list above (no. 1-6), the elements of the chimeric polypeptide are thus preferably shown from the N terminus (left) to the C-terminus (right).

Preferably, the individual parts of different origin of the chimeric polypeptide, when embedded in the plasma membrane of cells, are provided in the same position and/or substantial orientation as in the original protein from which sequence parts were taken. Accordingly, the chimeric polypeptide is a type 1 single pass transmembrane receptor. Preferably, the N- and C-termini of the sequence stretch that substantially corresponds to the intracellular sequence of a receptor B correspond to the corresponding termini and/or orientation as found in the original receptor B. The same applies in analogy to sequences that are substantially identical to sequences of a receptor A. Preferably, only one transmembrane domain is present, which preferably separates the intracellular parts from extracellular parts of both original receptors A and B. In other words, the transmembrane domain is positioned appropriately. For example, if the chimeric receptor also comprises the intracellular part of a receptor A, the transmembrane domain is located on the amino acid sequence so that also in the chimeric polypeptide the intracellular part of receptor A is on the intracellular side of the chimeric polypeptide.

The reference receptors A and B are preferably of a natural origin. They may be as already reported, or they may be receptors that still will be discovered in the future, and to which the principle of the present invention can be applied. Of course, receptor A is selected in dependence of the purpose of the screening method, that is, the target, for which an active agent is sought. Accordingly, receptor A and receptor B may independently be isolated from any organism, in particular animals or humans. Preferably, the receptors A and B are, independently, human, or mammal animal receptors. According to an embodiment, receptors A and B are independently as present in a human, simian, rodent, ungulate, carnivore, bird, reptile, amphibian and/or insect. Receptors found in humans, rodents and domesticated animals, such as pets and livestock are preferred.

The chimeric polypeptide of the present invention thus comprises at least stretches (or, for example in case of the first part, a full length receptor A) of a naturally occurring receptor, or comprises sequence stretches which may be composed of stretches of different naturally occurring receptors.

Receptors A and B may also be referred to as “reference receptors” or “original receptor”, because, preferably, the respective part of the chimeric polypeptide of the invention stems from and/or is substantially identical to at least a portion of a naturally occurring receptor and the latter is thus the basis of a comparison. However, as mentioned above, in the amino acid sequences (and the encoding nucleotide sequences) of the invention, the original sequences may be modified for any particular purpose, in order to provide variants or sequences with similarity to the original reference receptor, depending on the desired properties of the final polypeptide.

The transmembrane domain, and the nucleotide sequence encoding it, may again be of any origin, that is, isolated from any organism having transmembrane protein domains, for example the organisms mentioned above. Furthermore, natural or artificial variants may be used.

According to an embodiment, the amino acid sequence of said first part is taken from and/or substantially identical to a continuous stretch of at least 80, 100, 120, 150, 170, 190 and most preferably at least 200 continuous amino acid moieties of the amino acid sequence of said receptor A.

According to an embodiment, the amino acid sequence of said second part is taken from and/or substantially identical to a continuous stretch of at least 200, 250, 350, 400, 450, 470, 500, and most preferably at least 520 continuous amino acid moieties of the amino acid sequence of said receptor B.

In other words, the compared sequences (first part to receptor A; second part to receptor B) encompass at least one continuous stretch preferably having at least the above indicated preferred lengths.

The chimeric polypeptide of the present invention may comprise further amino acid sequences or may be further modified, for example in vivo and/or in vitro, for example by chemical modification. For example linker sequences, cell-compartment targeting sequences, sequences with protease cleavage sites, marker sequences, oligomerization domains, effector protein binding domains, domains assisting in protein isolation, catalytically active domains, glycosylation, just to mention a few, may be present on or be part of the chimeric polypeptide of the invention. Additional amino acid sequences may be provided terminally or between other sequence parts constituting the chimeric polypeptide of the invention. This applies, for example, to possible linker sequences. Said additional amino acids and/or amino acid sequences may be present also in the embodiments numbered 1-4 above. The additional domains or sequences may be encoded, for example, by continuous reading frame of the nucleotide sequence encoding the chimeric polypeptide of the invention and may or may not be removed in vitro, or, in vivo, for example by pre-mRNA cleaving, RNA splicing, posttranscriptional modifications, protein modification by protein splicing, proprotein convertase and signal peptide peptidase, for example.

The chimeric polypeptide of the invention may be substantially formed by a continuous amino acid sequence, in which each amino acid residue is connected to the respective neighbour(s) by a peptide bond (a fusion protein). The separate domains may, of course, contain additional amino acid sequences as mentioned above (linkers, etc.).

Alternatively, the chimeric polypeptide of the invention may comprise two or more separate amino acid sequences forming separate protein domains, which may be connected covalently or non-covalently, to form a complex comprising separate protein units. For example, one, two or all three individual parts and/or domains of the chimeric polypeptide (extracellular, transmembrane and cytoplasmic domains) may be connected to the respective neighbouring domain by way of one or more disulfide bonds.

The present invention provides one or more nucleotide sequences encoding the chimeric polypeptide of the present invention. According to an embodiment, the present invention provides a nucleic acid comprising a single continuous or several separate nucleotide sequences encoding the chimeric polypeptide of the invention. Preferably, the nucleic acid molecule may comprise a first sequence encoding at least the extracellular, ligand-binding portion of a receptor A, a second sequence encoding at least the intracellular, signaling kinase portion of a receptor B and, if not yet comprised in between said first and second sequences, a third sequence encoding a transmembrane domain. Preferably, said first, second and, optionally, third sequences are provided in the form of an overall continuous coding sequence. As indicated above, the continuous coding sequence may also encompass and/or encode further amino acids or sequences, as exemplified elsewhere in this specification. The nucleic acid may further comprise a promoter sequence, such as one of those specified in more detail below, which controls expression of said the sequence(s) encoding said chimeric polypeptide.

The attached sequence listing discloses nucleotide and amino acid sequences, respectively, of the following exemplary fusion proteins in accordance with various preferred embodiments of the present invention:

The fusion of full length hTNFR1 with the truncated, cytoplasmic, tyrosine kinase domain of mouse mPDGFR: SEQ. ID. NO.: 1 and 2.

The fusion of truncated (extracellular and transmembrane domains) human TNFR1 (hTNFR1) with the truncated, cytoplasmic, tyrosine kinase domain of mouse PDGFR (mPDGFR): SEQ. ID. NO.: 3 and 4.

The fusion of truncated hTNFR1 (extracellular domain) with the truncated, transmembrane and cytoplasmic tyrosine kinase domain of mPDGFR: SEQ. ID. NO.: 5 and 6.

The fusion of truncated hTNFR1 (extracellular and transmembrane domains) with the truncated, cytoplasmic, tyrosine kinase domain of hEGFR: SEQ. ID. NO.: 7 and 8.

The fusion of truncated hTNFR1 (extracellular domain) with the truncated, transmembrane and cytoplasmic tyrosine kinase domain of hEGFR: SEQ. ID. NO.: 9 and 10.

The fusion of full length DR3 with the truncated, cytoplasmic, tyrosine kinase domain of mouse mPDGFR: SEQ. ID. NO.: 11 and 12.

The fusion of truncated (extracellular and transmembrane domains) FAS with the truncated, cytoplasmic, tyrosine kinase domain of mouse PDGFR: SEQ. ID. NO.: 13 and 14.

The fusion of truncated FAS (extracellular domain) with the truncated, transmembrane and cytoplasmic tyrosine kinase domains of mouse PDGFR: SEQ. ID. NO.: 15 and 16.

The fusion of full length FAS with the truncated TNFR1 death domain and with the truncated, cytoplasmic, tyrosine kinase domain of mouse mPDGFR: SEQ. ID. NO.: 17 and 18.

The fusion of truncated (extracellular and transmembrane domains) FAS with the truncated, cytoplasmic domain of TNFR1, with the truncated cytoplasmic domain, tyrosine kinase domain of mouse PDGFR: SEQ. ID. NO.: 19 and 20.

The fusion of full length TNFR2 with the truncated TNFR1 death domain and with the truncated, cytoplasmic, tyrosine kinase domain of mouse mPDGFR: SEQ. ID. NO.: 21 and 22.

The present invention encompasses a nucleotide sequence according to any one of SEQ. ID. NO.: 1, 3, 5, 7, 9, 11, 13, 15, 17, 19, and 21, and nucleotide sequences encoding polypeptides as defined below.

The present invention also provides chimeric polypeptides comprising an amino acid sequence according to any one of SEQ. ID. NO.: 2, 4, 6, 8, 10, 12, 14, 16, 18, 20, and 22, and a polypeptides having at least 60% or more sequence identity (the indications concerning sequence identity given above apply independently) with any one sequence selected from SEQ. ID. NO.: 2, 4, 6, 8, 10, 12, 14, 16, 18, 20, and 22.

When embedded in a membrane, preferably the plasma membrane, of a cell and under physiological conditions, the chimeric polypeptide of the invention as disclosed and described above is preferably capable of binding, preferably on the outer cell surface, a ligand that under natural and/or physiological conditions binds to receptor A. Following binding, the chimeric polypeptide is preferably capable of generating an RTK-like or tyrosine kinase mediated signal inside the cell.

Without wishing to be bound by theory, if the receptor A is selected from receptors of the TNFRSF, for example from TNFRs, the chimeric polypeptide is supposed to oligomerize, preferably trimerize, and to induce tyrosine kinase trans- and/or autophosphorylation and to thereby induce RTK-mediated signalling. When there is ligand binding and/or RTK-mediated signalling the chimeric receptor is in an active condition, which is different from the condition when there is no ligand binding, for example.

Cellular conditions affected by RTK-signalling may be recognised in screening methods and enable thus the detection of a binding and/or activation of the chimeric receptor of the invention. Since at least the extracellular, binding portion of the chimeric receptor is taken from and/or substantially identical to at least the extracellular, binding portion of a TNFRSF receptor, any compound binding to the receptor of the invention can be expected to be active on the original receptor (receptor A).

According to an embodiment, said cellular condition is at least partly dependent on an activity of said chimeric polypeptide. The chimeric polypeptide may thus exist in an active form and in inactive form. Furthermore, in cells containing several chimeric receptors, some of the receptors may be active and others inactive, in particular in dependence of the concentration of an active agent. The latter situation results in a partial activity, so that the screening method is preferably suitable to quantify activity on a substantially continuous scale.

Preferably, the “activity” of the chimeric polypeptide is a signalling activity, which is generally the consequence of ligand binding and the oligomerization of receptor subunits as discussed elsewhere in this specification. The oligomerization following ligand binding results in activation of tyrosine kinase activity, which in turn affects the cellular condition.

According to an embodiment, an agent affects the activity of a receptor if it affects a status of signalling of the receptor. The “status of signalling” preferably refers to the presence, absence or degree of signalling activity, of a receptor, for example all receptors of the same type of a cell. For example, an agent is active if it stops a receptor that is signalling, or if it induces signalling of a receptor that was not signalling before. The term “signalling” is understood as transducing or transmitting any kind of cellular signal to the intracellular and/or cytoplasmic part of the cell. As the skilled person understands, a signal may involve a cascade of intracellular and molecular events, in particular chemical reaction, which result in the change of the cellular condition of the cell. In particular, the concentration of second messengers or other cellular components may change.

Preferably, an activity of the chimeric receptor is thus equivalent to tyrosine kinase activity, preferably as specified elsewhere in this specification.

The present invention provides a method of screening compounds and/or compositions of matter exhibiting and/or exerting an activity, in particular a biological activity, on a receptor, in particular a receptor A as defined herein. For the purpose of this specification, this is equivalent to saying that the invention provides a method of screening for (or of) agents that affect the activity of a receptor A. Such compounds and/or compositions of matter may be referred to herein as “active agents”, or simply “agents”. Preferably, activity refers to cell signalling activity. A “candidate agent” may be any substance of matter. For example, isolated chemical compounds (molecules) or compositions of matter, such as composition of compounds, for example extracts, such as reaction mixtures, plant extracts and the like. The compound may be a macromolecule. In principle, the only limitation with respect to the “agent to be screened” one can spontaneously think of is that it can be added to a well plate of a microtiter plate comprising the cells.

Active agents, as understood in this specification, encompass and preferably are agonists, antagonists and modulators, for example. The agents may be binding to orthosteric and/or allosteric sites of receptor A and/or the polypeptide of the invention. The terms agonists and antagonists encompass natural ligands—endogenous (ant)agonists—as well as exogenous (ant)agonists.

Modulators are generally compounds that act in a modulating manner in conjunction with an agonists or antagonist, in particular with a natural ligand. Modulators may again be classified as “active modulators”, which encompass and preferably consist of “inhibitors”, “activators” and/or “neutral modulators” of receptor A. “Neutral modulators” are chemical entities that bind to the target without direct modulation of its function, but they prevent the binding of the natural ligand and/or other modulators or bioactive principles that share the same binding site on the target receptor, and in that way indirectly affect its activity and/or modulation.

According to an embodiment, the invention provides a method for screening active agents of a receptor A selected from receptors of the TNFRSF.

For example, if receptor A is selected from receptors of the TNFRs, an active agent may be an agent that prevents binding of the corresponding TNF. Such an active agent can then be used to prevent TNFR mediated signalling.

According to an embodiment, an agent affects the activity of a receptor if it affects a status of signalling of the receptor. The “status of signalling” preferably refers to the presence, absence or degree of signalling activity, of a receptor, for example all receptors of the same type of a cell. For example, an agent is active if it stops a receptor that is signalling, or if it induces signalling of a receptor that was not signalling before. The term “signalling” is understood as transducing or transmitting any kind of cellular signal to the intracellular and/or cytoplasmic part of the cell. As the skilled person understands, a signal may involve a cascade of intracellular and molecular events, in particular chemical reaction, which result in the change of the cellular condition of the cell. In particular, the concentration of second messengers or other cellular components may change.

In the screening method of the invention, an automated apparatus system is preferably used. Such a system may allow one or more or all of the following: high throughput screening; analysis of host cells containing reporter molecules (for example, fluorescent or luminescence reporter molecules); treating the host cells with one or more candidate agents; treating the host cells with one or more agents of known activity, such as the natural ligand; imaging and recording numerous cells at once, for example with fluorescence or luminescence optics; converting the optical information into digital data; utilizing the digital data to determine the concentration, and/or the activity of the reporter molecules in the cells and/or the distribution of the cells; and interpreting that information in terms of a positive, negative or null effect of the candidate agent on the at least one cellular characteristic.

The screening methods of the invention preferably use cells containing, preferably embedded in a membrane, the chimeric polypeptide, and/or expressing a nucleotide sequence encoding the chimeric polypeptide of the invention. These cells are also referred to as host cells.

The cells may for example be a mammalian cell such as for example a cell of bovine, porcine, rodent, monkey or human origin. The mammalian cell may for example be any one of the group consisting of a HeLa cell, a U2OS cell, a Chinese hamster ovary (CHO) cell, a CHO-KL cell, a HEK293 cell, a HEK293T cell, an NSO cell, a CV-1 cell, an L-M(TK-) cell, an L-M cell, a Saos-2 cell, a 293-T cell, a BCP-1 cell, a Raji cell, an NIH/3T3 cell, a C127I cell, a BS-C-1 cell, an MRC-5 cell, a T2 cell, a C3H10T1/2 cell, a CPAE cell, a BHK-21 cell, a COS cell (for example, a COS-1 cell or a COS-7 cell), a Hep G2 cell, and an A-549 cell. Such cells and other suitable cells are publicly available, for example from commercial sources such as the American Type Culture Collection (ATCC), the European Collection of Cell Cultures (ECACC) and/or the Riken Cell Bank (Tokyo, Japan).

The cells may comprise and/or be transfected to express an expression vector comprising any one of the nucleic acids and/or nucleotide sequences as disclosed herein. Expression of the nucleic acid may be driven by a constitutive or inducible promoter. Typically, the promoter is positioned upstream of the nucleic acid/nucleotide sequence encoding the polypeptide to allow transient or stable expression, for example in mammalian cells. The expression vector may comprise a Tet-ON® inducible expression system. Use of an inducible expression system allows higher levels of the polypeptide of the invention to be present when desired or required. Expression may be inducible for example upon addition of doxycyclin, tetracycline, or an analogue of either, such in a mammalian cell for example a CHO cell or other cells disclosed herein. The nucleic acid/nucleotide sequence, expression vector or polypeptide may be transiently or stably transfected into the host cell.

The cells are preferably provided at an approximately determined number in the wells of a microtiter plate. Each well and the cells plated therein thus constitute a sample. Cells may be added or plated in the wells of a microtiter plate in an automated manner.

The screening method of the invention comprises the step of exposing a candidate agent to be screened to said cell. As mentioned above, this may be done in an automated manner. Preferably, the present invention provides the step of adding said candidate agent at different concentrations to different wells of a microtiter plate, preferably in an automated manner.

The screening method of the invention comprises the step of measuring a physical, biological and/or chemical value that is associated with and/or corresponds to a cellular condition of said cells. Said cellular condition is preferably an intracellular condition.

Preferably, said cellular condition is affected if said candidate agent is an active agent, in particular of said receptor A. According to an embodiment, said cellular condition is at least partly dependent of and/or affected by an activity and/or condition of said chimeric polypeptide. For example, said cellular condition is dependent on and/or affected by the presence or absence of a specific form of oligomerization of the intracellular and/or extracellular components of said chimeric polypeptide, and/or for example on the RTK-activity of the intracellular domains of the chimeric receptor, and/or of ligand binding at the extracellular portion of the chimeric receptor.

According to an embodiment, binding of an active agent to said chimeric polypeptide may at least to some extent induce and/or prevent oligomerization of a plurality of said chimeric polypeptides and/or wherein said oligomerization induces a kinase activity of said intracellular kinase portion of the chimeric polypeptide.

According to an embodiment, the method of screening further comprises the steps of exposing said cells to a control agent. The control agent preferably has a known, reported and/or established effect on the activity of said receptor A. The method preferably comprises determining the capacity of said candidate agent to modulate activation and/or binding of said control agent to said chimeric polypeptide. Preferably, a candidate agent affects the activity of said receptor A if it affects an effect of said control agent on the activity of said chimeric polypeptide. Examples of such active agents are allosteric modulators, such as positive or negative allosteric modulators (PAMs and NAMs).

The control agent may be selected from orthosterically or allosterically binding ligands of receptor A. For example, the control agent is selected from natural ligand(s) of the receptor A. The control agent is an agent whose concentration-response curve is reported or can conveniently be established by the screening method of the invention, in particular by adding different (e.g. increasing) concentrations of the agent to the cells and measuring the intensity of the physical, biological and/or chemical value. In this way, EC values can be established for the control agent (ECO-EC100), indicating the minimum concentrations to obtain a signal that is distinguishable from baseline and the concentration that is needed to obtain a maximum signal/value. The control agent may be added at concentrations corresponding to EC values that are covered by the ranges EC5-100, EC5-97, EC10-90, EC20-80, for example. Accordingly, the method of the invention may be used to screen for modulators, which do not directly activate or inhibit a receptor, but which modulate the receptor activity in response to a directly activating or inhibiting agent, such as a natural ligand.

For example, the control agent (for example, the natural ligand or the ligand of reported effect) may be added in two- or more addition protocol, for example a co-addition protocol. In this way, inhibitors or activators of receptor A may be found, for example.

According to an embodiment, the method of the invention comprises the step of measuring a physical, biological and/or chemical value that is associated with a cellular condition of said cells.

According to a preferred embodiment, said cellular condition is affected by the activity and/or absence of activity of the intracellular kinase domain of said chimeric polypeptide. According to an embodiment, said cellular condition is at least partly dependent on the presence of activity, absence of activity, and/or extent of activity of the intracellular kinase domain of said chimeric polypeptide. As mentioned above, said tyrosine kinase activity may, in turn, be dependent on the binding of an active agent and/or oligomerization or absence of oligomerization of the extracellular and/or intracellular domains of the chimeric polypeptide.

According to an embodiment, said physical, biological and/or chemical value that is associated with and/or corresponds to a cellular condition is fluorescence and/or luminescence, in particular bioluminescence.

It is noted that the expression “physical, biological and/or chemical value” refers to any measurable signal produced by the cells following binding and/or modulation of receptor activity. Presently, many reporting systems produce light, which can be conveniently detected using appropriate equipment. Light produced by a reporting system may be produced by a luminescent protein, possibly under consumption of a particular chemical substrate that is specifically added to the cells. In this regard, the light amount is indeed all of the above: a physical value (light intensity), a biological value (reflecting bioluminescent activity) and a chemical value (reflecting substrate consumption).

One could also measure other parameters or signals, as reporting systems producing radioactivity (less frequently used today) or other markers (substrate consumption, product generation, etc.). The quantification of such signals can generally in all cases be considered as the measurement of a physical, biological and/or chemical value. Measurements are generally made with the corresponding equipment.

Preferably, a reporting system produces a signal in dependence of a cellular condition, such as the concentration of a cellular component, for example a second messenger.

Preferably, the cellular condition is an intracellular condition.

Activated tyrosine kinase domains of RTKs, one of which is substantially part of the chimeric polypeptide, are reported to be phosphorylated or active on a variety of signaling proteins, and, depending on the specific signal transduction pathway induced, to lead to the recruitment of adapter, or to the release of intracellular secondary messengers, such as Ca²⁺, inositol phosphate (IP1) and inositol triphosphate (IP3). Therefore, according to an embodiment, the intracellular condition is concentration or a change in the concentration of one or more selected from: free intracellular Ca²⁺, inositol phosphate (IP1) and inositol triphosphate (IP3). According to an embodiment, said cellular condition is the degree in phosphorylation or recruitment of adapter proteins.

Numerous reporting systems sensing changes in phosphorylation or recruitment of adapter proteins or in intracellular Ca²⁺, inositol phosphate (IP1) and/or inositol triphosphate (IP3) concentrations are available to the skilled person. For example, changes in phosphorylation can be measured by flow cytometry using specific monoclonal antibodies recognizing phosphorylated amino-acids or protein sequences containing phosphorylated amino-acids.

For example, reporting systems are available producing measurable physical values in dependence of free intracellular Ca²⁺ concentration.

For example, aequorin is a photoprotein isolated from luminescent jellyfish and is composed of two distinct units, the apoprotein apoaequorin and coelenterazine, a luciferin. The two components of aequorin reconstitute spontaneously, forming the functional protein. The protein bears several binding sites for Ca²⁺ ions, which, when bound, trigger the protein to undergo a conformational change. As the excited protein relaxes to the ground state, blue light (wavelength=469 nm) is emitted. Therefore, according to an embodiment, the cells of the present invention preferably express apoaequorin. For example, the cells are transfected to express apoaequorin. In this case, the screening method of the invention preferably comprises the step of adding a luciferin, in particular coelenterazine to the cells. In this embodiment, the light emitted by aequorin (luminescence) constitutes the physical value that is measured in the method of the invention. More specifically, said physical value is bioluminescent light having a wavelength having a maximum intensity in the wavelength range of 400-540 nm, preferably 440-500 nm, most preferably about 460-480 nm. Aequorin emits blue light (wavelength=469).

The skilled person may, of course, select any other indicator of intracellular Ca²⁺ concentration, such as for example, the Fluo-4 No Wash (NW) dye mix commercially obtainable from Molecular Probes, USA. In this and other systems, intensity and/or wavelength of fluorescent light is dependent on intracellular free Ca²⁺ concentration, said fluorescent light thus forming a measurable and interpretable physical value.

The expressions “associated with” and/or “corresponding to” for the purpose of the present specification have their general meaning. They thus reflect any kind of correlation and/or link between the cellular condition and the physical, biological and/or chemical value that can be measured. The strength of the signal is generally associated with (that means correlates in some way with) the cellular condition (e.g. second messenger concentration). For example, in the case of light produced by aequorin, the intensity of the light correlates with intracellular, free Ca²⁺ concentration, so that the measurement of a light intensity can be interpreted as a particular, approximate concentration of free Ca²⁺.

The method of the invention preferably comprises the step of determining, from the value measured in the preceding step, if said candidate agent is an agent exhibiting an activity on said receptor A. In this regard, the determination step generally involves the comparison of the value of the actually measured physical, biological and/or chemical signal in accordance with the method of the invention to a basic value. The basic value is determined, for example, in the absence of said candidate agent (the negative control). The basic or negative control value may be determined beforehand, that is, before running the method of the invention. In FIGS. 8a and 8b, the very left side, an isolated data point in the graphs corresponds to such a basal or basic value. Generally, a threshold value is generated or determined, which is sufficiently far away from the negative control value so as to account for natural variations occurring in the signal measurement. The methods of determining such threshold values, which also relates to the avoidance of false positives, can be established by the person skilled in the art. The same applies with respect to the statistics that one may use to increase the probability that a given measured deviation from the negative control or from the threshold value corresponds indeed to a “hit” (an active agent). In particular, measurements may be repeated and the mean of several separate measurements may be used for purpose of comparison and thus, determining if an agent is considered as an active agent.

From the above it becomes clear that the administration of a candidate agent, if it affects the cellular condition of the cells, should induce the reporting system to produce a detectable change of the physical, biological and/or chemical value. The candidate agent is then considered an active agent (a hit). In accordance with an embodiment, said candidate is an active agent of said receptor A, if it affects said cellular condition of said cells.

The invention is disclosed in further detail in the following examples, which are in no way intended to limit the scope of the present invention.

### EXAMPLES

### Examples 1-3

### Preparation of Constructs and Transfection Vectors of Chimeric TNFR1-PDGFR in Accordance with Embodiments of the Invention

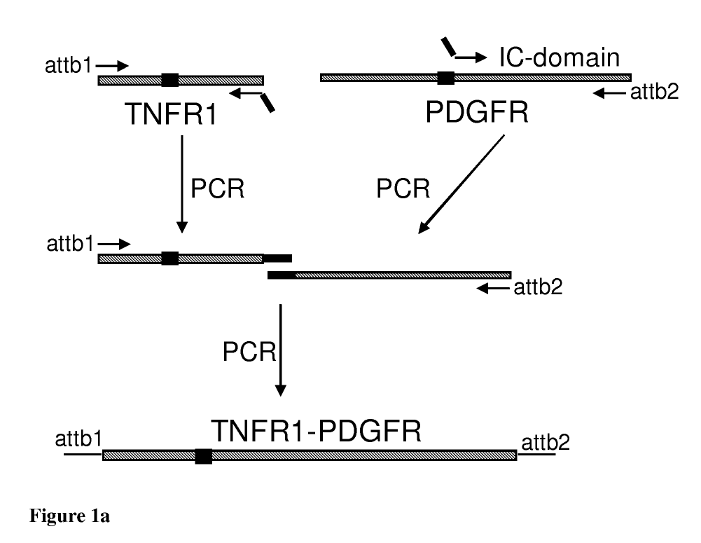

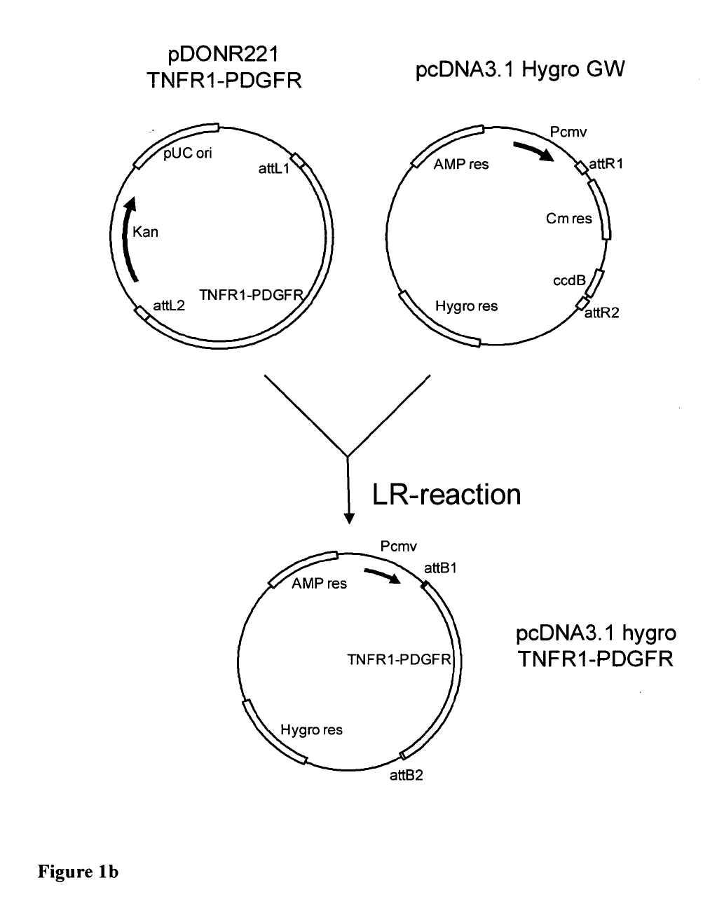

Gene constructs (Table 3) comprising TNFR1 DNA (Access no.: NM_{—}001065.2) fused to mouse PDGFRb DNA (Access no.:NM_{—}008809.1) were prepared as schematically shown in FIG. 1a.

TABLE 3
TNFR1-PDGFRb constructs

| Construct/ | SEQ. ID. | | |
|---|---|---|---|
| Example no. | NO.: | TNFR1 domains | PDGFR domains |
| 1 | 1, 2 | full length | cytoplasmic domain (cp) |
| | | (fl) (bp 282-1646) | (bp 1810-3435) |
| 2 | 3, 4 | extracellular (ex) and | cytoplasmic domain (cp) |
| | | transmembrane (tm) | (bp 1810-3435) |
| | | (bp 282-980) | |
| 3 | 5, 6 | extracellular | transmembrane (tm) and |
| | | (bp 282-914) | cytoplasmic domain (cp) |
| | | | (bp 1717-3435) |

For preparing these constructs and expression vectors, standard cloning techniques were used according to manufacturer's instructions.

The resulting PCR product encoding the chimeric receptor was inserted into the pDONR221 vector of Invitrogen using the Gateway BP Clonase® enzyme mix (Invitrogen), according to the manufacturer's protocol.

To generate the appropriate expression vector the Gateway cassette® (Invitrogen) was inserted into the ECORV site of the pcDNA3.1 hygromycin vector (Invitrogen), using standard cloning techniques. The chimeric receptor DNA was introduced into the expression vector pcDNA3.1 hygro GW using the LR Clonase® II enzyme mix of Invitrogen (FIG. 1b), according to the manufacturer's protocol, yielding the expression construct pcDNA3.1 hygro TNFR1(fl)-PDGFR(cd) vector.

### Example 4

### Transfection of HEK293T Aequorin Cells and Expression of the Chimeric Receptors

HEK293T stably expressing Apoaequorin were generated using standard cloning techniques. The HEK293T cells expressing Apoaequorin (“Aequorin cells”) were then further transfected as described in Examples 1-3 so as to express the chimeric receptors 1-3 as listed in Table 3.

In particular, the HEK293T Apoaequorin cells were transfected with pcDNA3.1 hygro TNFR1-fl-PDGFR-cd vector as prepared in Example 1 using Optifect™ Transfection Reagent (Invitrogen), according to the manufacturer's protocol.

Cell surface expression of the chimeric receptors comprising full length TNFR1 and the cytoplasmic domain of PDGFR was detected by flow cytometry. Briefly, cells were harvested and incubated with a monoclonal antibody directed against TNFR1 (MAB225, R&D Systems) or an isotype matched mouse IgG (both purchased from R&D systems, Minneapolis, Minn., USA). Both antibodies were used at a final concentration of 1 μg/ml. Cells were washed twice and incubated with Cy3-conjugated F(ab′) fragments of a donkey anti-mouse polyclonal antibody (Jackson ImmunoResearch, Westgrove, Pa., USA) at a final concentration of 0.2 μg/ml. Subsequently, cells were washed twice and resuspended in a final volume of 500 μl. All antibody incubations were performed in flow cytometry buffer (PBS containing 5% FBS and 0.01% sodium azide) for 20 minutes at 4° C. Flow cytometry was performed using a FACSCalibur and results were analyzed using Cellquest software (BD Biosciences, San Jose, Calif.).

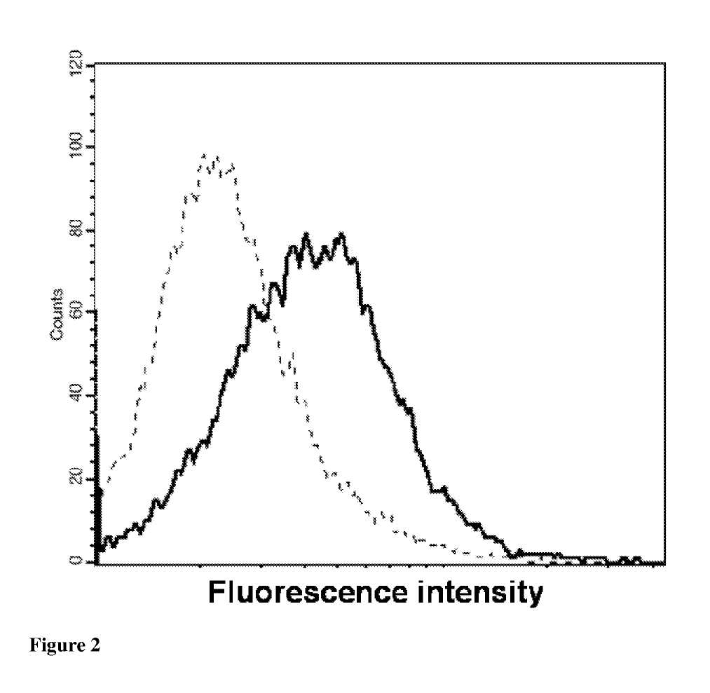

The flow-cytometrical results are shown in FIG. 2, where the black solid line corresponds to anti-TNFR1 mAb staining and the dotted line corresponds to the values obtained with the isotype control.

These results show that the extracellular domain of TNFR1 of the chimeric receptor is found at the surface of the transfected HEK293T Aequorin cells.

### Example 5

### Detection of Intracellular Calcium Levels in an HTS Setting

The property of aequorin to produce light in dependence of intracellular free Ca²⁺ ions is described above.

HEK293T cells expressing Apoaequorin and the chimeric receptor (Example 4) were plated in 384-well plates at a concentration of 12500 cells per well in a final volume of 50 μl. The next day culture supernatants were removed and 25 μl labeling buffer (DMEM:F12 plus 0.1% BSA) containing 2.5 μM Coelenterazine h (Dalton Pharma services), was added. Cells were incubated at room temperature for 6 hrs. A FDSS7000 reader from Hamamatsu (Japan) was used to examine intracellular calcium levels. This instrument is designed for high throughput screening and high throughput analysis. The instrument features include detection with a camera of fluorescence or luminescence and automatically converts fluorescence or luminescence signals into numeric data. This digital data is then used to determine the concentration of calcium inside the analyzed cells. The information is automatically analyzed in terms of a positive, negative or null effect of each test compound being examined. This system allowed differences between untreated and treated cells to be measured, for example by measuring the calcium flux in cells.

After a baseline reading of 10 seconds, cells were incubated for 3 minutes with different doses of inhibitors or buffer controls. Subsequently, cells were stimulated with TNF and measurements were continued for another 10 minutes. The results were analyzed using the FDSS analysis software from Hamamatsu.

FIGS. 3a and 3b are dose response curves obtained by exposing the cells of Example 4 to increasing concentrations of TNF. FIG. 3a is established on the basis of the integration of the luminescence emitted in 10 minutes following administration of TNF (exposure time) in dependence of the applied TNF concentration (AUC), while FIG. 3b is established on the basis of intensity of the response in dependence of the applied TNF concentration (max-min). The concentration of TNF ranged from 50 ng/ml to 100 pg/ml. The results are representative of four independent experiments and the error bars represent the standard deviation of triplicate wells. FIG. 4 shows the individual traces of luminescent signal corresponding to the TNF concentrations ranging from 50 ng/ml to 100 pg/ml.

Table 4 below shows the EC50 and EC80 values determined on the basis of the results shown in FIGS. 3a and 3b.

TABLE 4
EC50 and EC80 TNF concentrations on the cells expressing the
chimeric TNFR1-PDGFR Receptor According to
an Embodiment of the Invention

| Method | EC50 | EC80 |
|---|---|---|
| AUC | 1.27 ng/mL | 2.9 ng/mL |
| Max-Min | 1.06 ng/mL | 3 ng/mL |

### Example 6

### Effect of TNFR1 and PDGFR Agents on the Calcium-Dependent Luminescence Signal in the Cells of the Invention in HTS

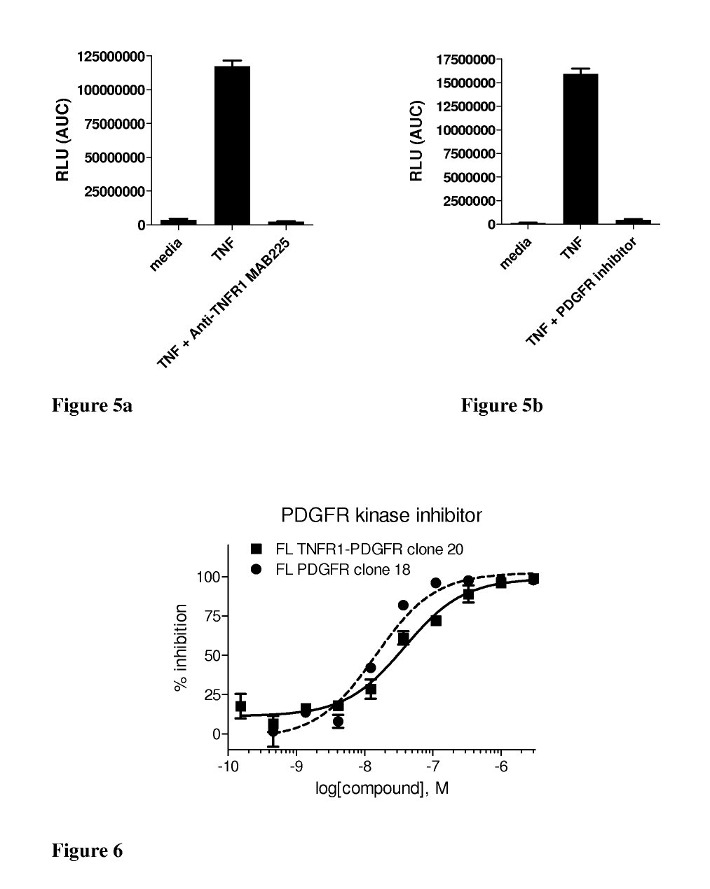

HEK293T Aequorin cells expressing fusion proteins as described in Example 4 were treated or not treated with 3 ng/ml of TNF and 300 ng/ml of an anti-TNFR1 antagonist monoclonal antibody (MAB225, R&D Systems) was added to half of the samples exposed to TNF. Following 10 minutes of exposure after TNF addition, the area under the curve was determined for each sample. The result is seen in FIG. 5a. As can be seen, the anti-TNFR1 antibody completely prevented TNF-induced signaling, that is, the increase of intracellular free Ca²⁺. This experiment shows that the constructs, chimeric polypeptides and cells of the invention are suitable in screening methods of agents exhibiting an activity on TNF receptors.

In another experiment, Aequorin cells of Example 4 exposed to 3 ng/ml of TNF were or were not exposed, besides TNF, to 4-(6,7-Dimethoxy-4-quinazolinyl)-N-(4-phenoxyphenyl)-1-piperazinecarboxamide, a PDGFR Tyrosine Kinase Inhibitor III of Calbiochem (USA). As can be seen from FIG. 5b, addition of 1 μM of inhibitor prevented the detection of intracellular calcium increase, showing that the TNF-dependent signal is mediated by the kinase domain of the chimeric receptor.

FIG. 6 shows that the PDGFR inhibitor as described above has equal inhibitory efficacy of Aequorin cells expressing the full-length PDGFR receptor and the TNFR1-PDGFR chimeric receptor. The concentration of PDGFR kinase inhibitor ranged from 3 μM to 0.45 nM. The results are representative of four independent experiments and the error bars represent the standard deviation of triplicate wells.

### Example 7

### Determination of Suitability for HTS

The “Z′-factor” of an assay is a statistical measure used to evaluate a high-throughput screening (HTS) assay. A score close to 1 indicates an assay is ideal for HTS and a score less than 0 indicates an assay to be of little use for HTS (see Zhang et al., 1999, J. Biomol. Screen. 4: 67-73). Four parameters needed to calculate the Z′-factor are: mean (μ) and standard deviation (σ) of both positive (p) and negative (n) control data (μₚ, σₚ, μₙ, σₙ, respectively). Using the formula:

Z′-factor=1−[3×(σₚ+σₙ)/|μₚ−μₙ|]

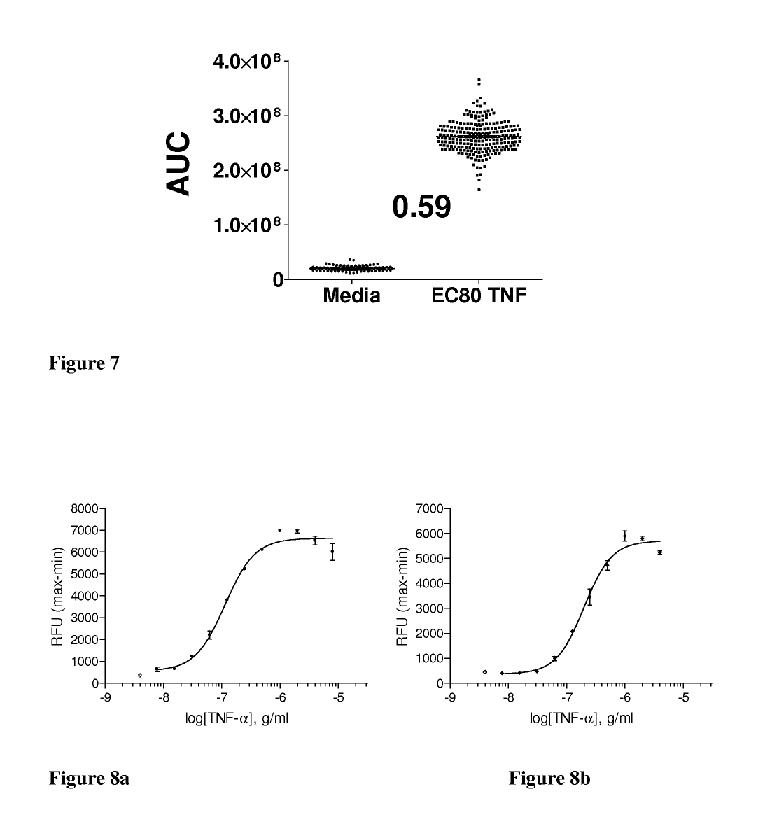

In order to determine the Z′-factor of the assay of the present invention, the cells of Example 4 above were plated in a 384-well plate as described above and exposed to EC80 of TNF (3 ng/mL) or to cell medium devoid of TNF (“Media”), and the area of curve was determined following 10 minutes of exposure. FIG. 7 is a scatter plot showing the calcium flux or concentration as area under the curve (AUC) of luminescence units for each sample. The Z′-factor for the assay results shown in FIG. 7 was calculated to be 0.59. The Z′-factor calculation demonstrated that the method of the invention is validated for use in HTS.

### Example 8

### Calcium Flux/Concentration Determined using the Fluo-4 Calcium Indicator

Fluo-4 AM is a cell-permeable fluorescent Ca²⁺ indicator that upon binding of calcium increases its fluorescence emission (excitation wavelength=494 nm and emission wavelength=516 nm). Therefore, fluorescent signal intensity correlates with intracellular calcium levels.

Intracellular calcium levels were determined using the Fluo-4 No Wash (NW) dye mix according to the manufacturer's recommendation (Molecular Probes, USA). In short, HEK293T cells transfected as described in Examples 1-3 so as to express the chimeric receptors 1-3 as listed in Table 3 were plated in 384-well plates at a concentration of 12500 cells per well in a final volume of 50 μl. The next day, culture supernatants were removed and 25 μl labeling buffer (1×HBSS, 20 mM Hepes), containing the Fluo-4NW dye mix and 2.5 mM probenecid, was added. Cells were incubated at 37° C. for 30 minutes, followed by 30 minutes at room temperature. Intracellular calcium levels were determined using a FLIPR Tetra (Molecular Devices, USA). After a baseline reading of 10 seconds, cells were stimulated with TNF and measurements were continued for another 10 minutes. The results were analyzed using the Screenworks software from Molecular Devices.

FIG. 8a shows a dose response curve of construct 2 established on the basis of the intensity of the fluorescent response (max-min) in dependence of the applied TNF concentration. The concentration of TNF ranged from 5 μg/ml to 4 ng/ml. The results are representative of four independent experiments and the error bars represent the standard deviation of triplicate wells.

FIG. 8b shows a dose response curve of construct 3 established on the basis of the intensity of the fluorescent response (max-min) in dependence of the applied TNF concentration. The concentration of TNF ranged from 5 μg/ml to 4 ng/ml. The results are representative of four independent experiments and the error bars represent the standard deviation of triplicate wells.

### Examples 9-10

### Preparation of Constructs and Transfection Vectors of Chimeric TNFR1-EGFR in Accordance with Embodiments of the Invention

Gene constructs (Table 5) comprising TNFR1 DNA (Example 1) fused to human EGFR DNA [NM_{—}005228.3] were prepared according to the same principle as schematically illustrated in FIG. 1a.

TABLE 5
TNFR1-EGFR constructs

| Construct/ | | | |
|---|---|---|---|
| Example no. | SEQ. ID. NO.: | TNFR1 domains | EGFR domains |
| 4 / 9 | 7, 8 | ex and tm (bp 282-980) | cp (bp 2251-3879) |
| 5 / 10 | 9, 10 | ex (bp 282-914) | tm and cp |
| | | | (bp 2182-3879) |

ex = extracellular;
tm = transmembrane;
cp = cytoplasmic

For preparing these constructsand, expression vectors, standard cloning techniques were used according to manufacturer's instructions.

The resulting PCR product encoding the chimeric receptor was inserted into the pDONR221 vector of Invitrogen using the Gateway® BP Clonase™ enzyme mix (Invitrogen), according to the manufacturer's protocol.

To generate the appropriate expression vector the Gateway cassette (Invitrogen) was inserted into the ECORV site of the pcDNA3.1 Hygro vector (Invitrogen). The chimeric receptor DNA was introduced into the expression vector pcDNA3.1 Hygro GW using the Gateway LR Clonase® II system of Invitrogen (FIG. 2), according to the manufacturer's protocol, yielding the expression construct pcDNA3.1 Hygro TNFR1(ex-tm)-EGFR(cd) vector.

Cells expressing the chimeric polypeptides of constructs 4 and 5, when exposed to the TNF ligand resulted in similar dose response curves as shown in FIGS. 3a and 3b. Furthermore, similar Z′-value as shown in FIG. 7 is determined.

### Example 11

### Preparation of a Construct and Transfection Vector of Chimeric DR3 (Full Length)-PDGFR (Cytoplasmic Domain)

Gene construct 6 (Table 6) comprising human DR3 DNA (TNFRS member 25), access no.: NM_{—}148965.1 fused to mouse PDGFRb DNA (example 1) was prepared according to the same principle as schematically illustrated in FIG. 1a.

For preparing these constructs and expression vectors, standard cloning techniques were used according to manufacturer's instructions.

The resulting PCR product (construct 6) encoding the chimeric receptor was inserted into the pDONR221 vector of Invitrogen using the Gateway BP Clonase® enzyme mix (Invitrogen), according to the manufacturer's protocol.

To generate the appropriate expression vector the Gateway cassette® (Invitrogen) was inserted into the ECORV site of the pcDNA3.1 hygromycin vector (Invitrogen), using standard cloning techniques. The chimeric receptor DNA was introduced into the expression vector pcDNA3.1 hygro GW using the LR Clonase® II enzyme mix of Invitrogen (according to the same principle as schematically illustrated in FIG. 1b for TNFR1), according to the manufacturer's protocol, yielding the expression construct pcDNA3.1 hygro DR3(fl)-PDGFR(cd) vector.

TABLE 6
DR3-PDGFR construct

| Construct/ | | | |
|---|---|---|---|
| Example no. | SEQ. ID. NO.: | DR3 domains | PDGFR domains |
| 6 / 11 | 11, 12 | fl (bp 89-1366) | cp (bp 1810-3435) |

ex = extracellular;
cp = cytoplasmic

### Example 12

### Transfection of HEK293T Aequorin Cells and Expression of the Chimeric DR3(fl)-PDGFR(cd) Receptor

The HEK293T cells expressing Apoaequorin (“Aequorin cells”) were transfected as described in Examples 1-3 so as to express the chimeric receptors DR3(fl)-PDGFR(cd) of Example 11.

In particular, the HEK293T Apoaequorin cells were transfected with pcDNA3.1 hygro DR3(fl)-PDGFR(cd) vector as prepared in Example 11 using Optifect™ Transfection Reagent (Invitrogen), according to the manufacturer's protocol.

Cell surface expression of the chimeric receptors comprising full length DR3 and the cytoplasmic domain of PDGFR was detected by flow cytometry. Briefly, cells were harvested and incubated with a PE-labeled monoclonal antibody directed against DR3 (clone JD3, BD Biosciences) or a PE-labeled isotype matched mouse IgG (both purchased from BD Biosciences). Subsequently, cells were washed twice and resuspended in a final volume of 500 μl. All antibody incubations were performed in flow cytometry buffer (PBS containing 5% FBS and 0.01% sodium azide) for 20 minutes at 4° C. Flow cytometry was performed using a FACSCalibur and results were analyzed using Cellquest software (BD Biosciences, San Jose, Calif.).

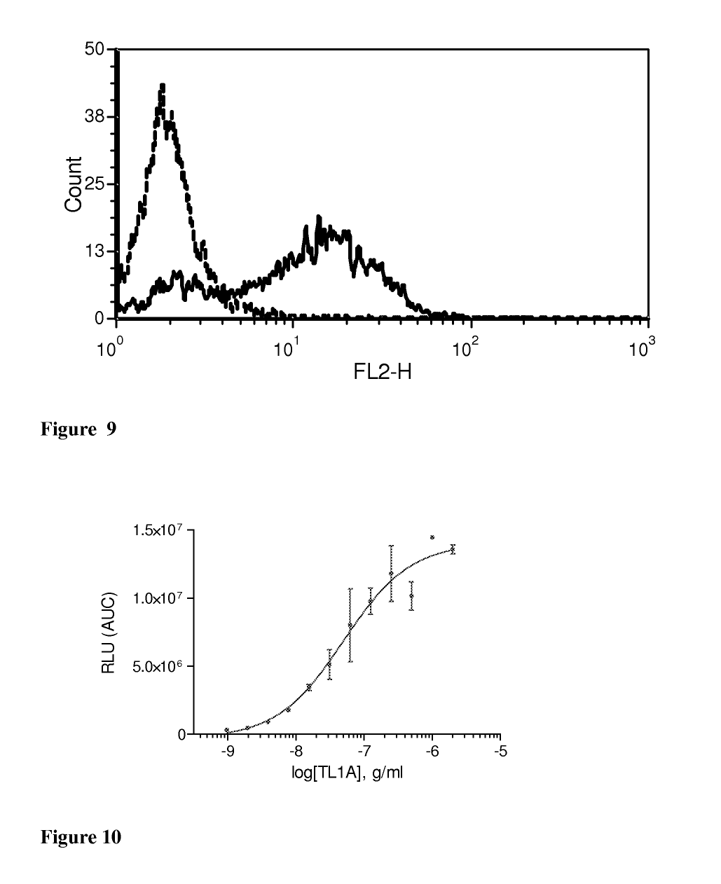

The flow-cytometrical results are shown in FIG. 9, where the black solid line corresponds to anti-DR3 mAb staining and the dotted line corresponds to the values obtained with the isotype control.

These results show that the extracellular domain of DR3 of the chimeric receptor is found at the surface of the transfected HEK293T Aequorin cells.

### Example 13

### Detection of Intracellular Calcium Levels in an HTS Setting of the Chimeric DR3(fl)-PDGFR(cd) Receptor

HEK293T cells expressing Apoaequorin and the chimeric DR3(fl)-PDGFR(cd) receptor (Examples 11 and 12) were plated in 384-well plates at a concentration of 12500 cells per well in a final volume of 50 μl. The next day culture supernatants were removed and 25 μl labeling buffer (DMEM:F12 plus 0.1% BSA) containing 2.5 μM Coelenterazine h (Dalton Pharma services), was added. Cells were incubated at room temperature for 6 h. A FDSS7000 reader from Hamamatsu (Japan) was used to examine intracellular calcium levels. This instrument is designed for high throughput screening and high throughput analysis. The instrument features include detection with a camera of fluorescence or luminescence and automatically converts fluorescence or luminescence signals into numeric data. This digital data is then used to determine the concentration of calcium inside the analyzed cells. The information is automatically analyzed in terms of a positive, negative or null effect of each test compound being examined. This system allowed differences between untreated and treated cells to be measured, for example by measuring the calcium flux in cells.

After a baseline reading of 10 seconds, cells were incubated for 3 minutes with buffer controls. Subsequently cells were stimulated with TL1A and measurements were continued for another 10 minutes. The results were analyzed using the FDSS analysis software from Hamamatsu.

FIG. 10 depicts the dose response curve obtained by exposing the cells of Example 12 to increasing concentrations of TL1A. FIG. 10 is established on the basis of the integration of the luminescence emitted in 10 minutes following administration of TL1A (exposure time) in dependence of the applied TL1A concentration (AUC). The concentration of TL1A ranged from 1 ng/ml to 2 μg/ml. The results are representative of three independent experiments and the error bars represent the standard deviation of triplicate wells.

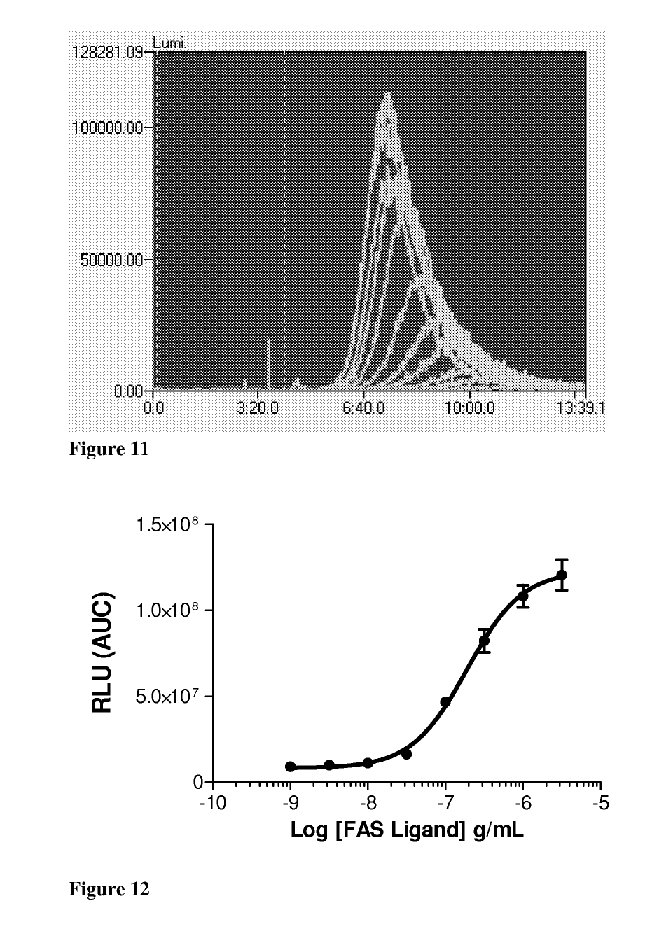

FIG. 11 shows the individual traces of luminescent signal corresponding to the TL1A concentrations ranging from 1 ng/ml to 2 μg/ml.

### Example 14

### Preparation of Constructs, Vectors and Transfected Cells of Different Embodiments Chimeric TNFRSF receptors According to the Invention

Gene constructs (Table 7) comprising human FAS DNA (Access no.: NM_{—}000043.4), or TNFR2 DNA (Access no.: NM_{—}001066.2), fused to mouse PDGFRb DNA Access no.:NM_{—}008809.1), were prepared according to the same principle as schematically illustrated in FIG. 1a.

TABLE 7
Further TNFRSF receptors-PDGFR constructs in accordance with further
embodiments of the present invention

| | SEQ. ID. | | |
|---|---|---|---|
| Construct | NO.: | TNFRSF receptor domains | PDGFR domains |
| 7 | 13, 14 | FAS ex and tm (pb 347-916) | cp (bp 1804-3435) |
| 8 | 15, 16 | FAS ex (pb 347-865) | tm and cp |
| | | | (bp 1717-3435) |
| 9 | 17, 18 | FAS fl (pb 347-1351) and | cp (bp 1804-3435) |
| | | TNFR1 DD (pb 1347-1614) | |
| 10 | 19, 20 | FAS ex and tm (pb 347-916) | cp (bp 1804-3435) |
| | | and TNFR1 cp (980-1646) | |
| 11 | 21, 22 | TNFR2 fl (pb 90-1472) and | cp (bp 1804-3435) |
| | | TNFR1 DD (pb 1293-1646) | |

Fl, full-length;
ex = extracellular;
tm = transmembrane;
cp = cytoplasmic;
DD = death domain

For preparing these constructs and expression vectors, standard cloning techniques were used according to the same principle as illustrated in the above examples.

The HEK293T cells expressing Apoaequorin (“Aequorin cells”) were transfected as described in Examples 1-3 and clones were selected so as to express the chimeric receptors described below:

FAS (ex and tm)—PDGFR (cd).

FAS (ex)—PDGFR (tm and cd).

FAS (fl)—TNFR1 (DD)—PDGFR (cd)

FAS (ex and tm)—TNFR1 (cp)—PDGFR (cp).

TNFR2 (fl)—TNFR1 (DD)—PDGFR (cp).

### Example 15

### Detection of Intracellular Calcium Levels in an HTS Setting of the Chimeric TNFRSF Receptors of Example 14

The clonal HEK293T cells expressing Apoaequorin and the chimeric receptors as described in Example 15 were plated in 384-well plates at a concentration of 12500 cells per well in a final volume of 50 μl. The next day culture supernatants were removed and 25 μl labelling buffer (DMEM:F12 plus 0.1% BSA) containing 2.5 μM Coelenterazine h (Dalton Pharma services), was added. Cells were incubated at room temperature for 6 h. A FDSS7000 reader from Hamamatsu (Japan) was used to examine intracellular calcium levels. After a baseline reading of 10 reads, cells were incubated for 4 minutes with buffer controls. Subsequently, cells were stimulated with appropriate agonist ligand FASL (Adipogen), or TNF (Peprotech) and measurements were continued until the response ended. The results were analyzed using the FDSS analysis software from Hamamatsu.

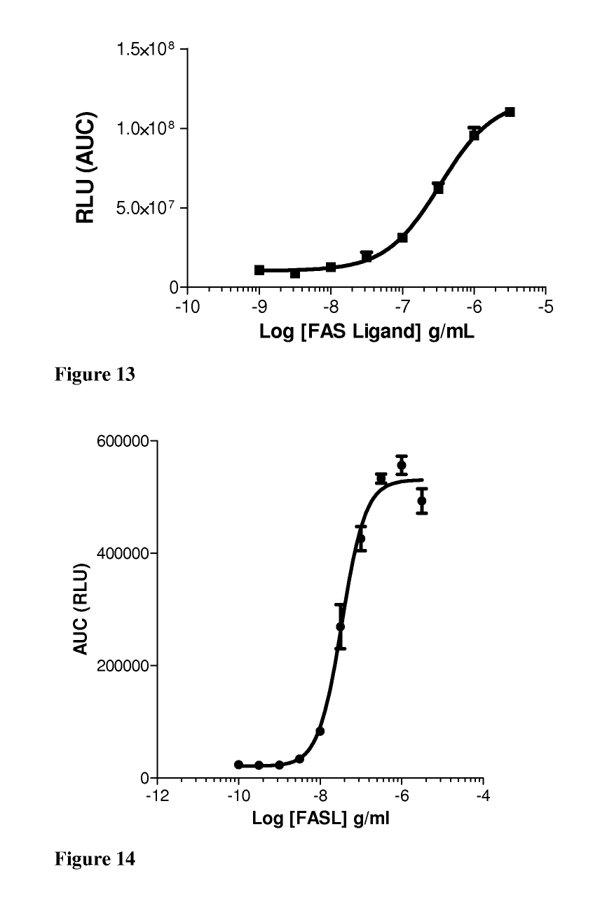

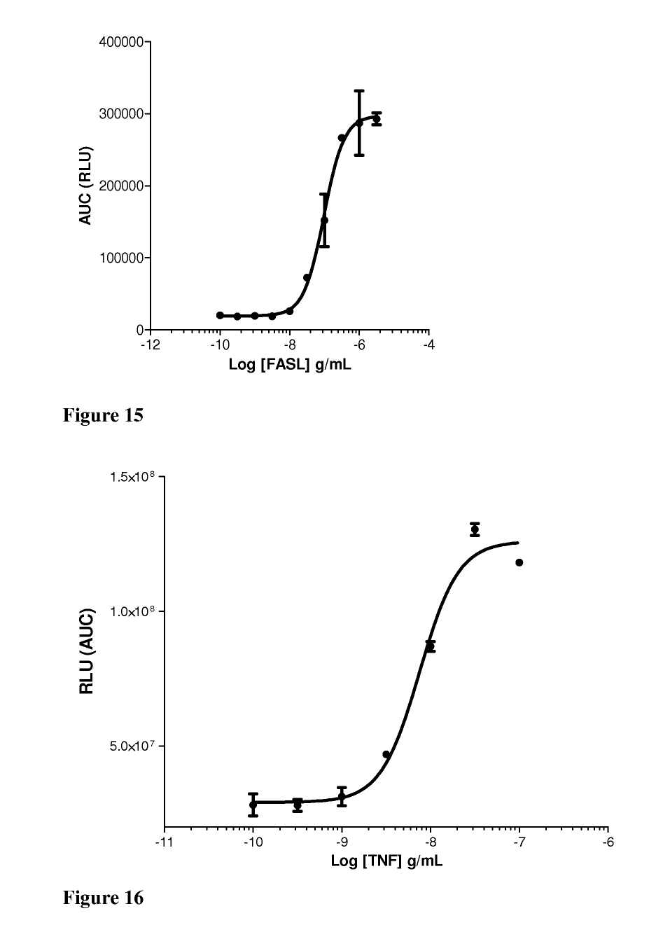

FIGS. 12-16 depict the dose response curve obtained by exposing the cells of to increasing concentrations of agonist ligands FASL, or TNF. FIGS. 12-16 are established on the basis of the integration of the luminescence emitted in 8 to 25 minutes following administration of agonist ligand (exposure time) in dependence of the applied agonist ligand concentration (AUC). The concentration of agonist ligand ranged from 10 pg/ml to 10 μg/ml. The results are representative of several independent experiments and the error bars represent the standard deviation of duplicate wells.

These examples show that various types of chimeric polypeptides as described in the present specification are suitable for drug screening or testing in an HTS setting. It is also noted that various combinations of the different constituent partials sequences yield chimeric receptors that retain the functions of ligand binding, oligomerization, and in the case of the RTK portion, tyrosine kinase activity specifically following ligand binding.

1. A chimeric polypeptide comprising:
a first part comprising an amino acid sequence that is substantially identical to the amino acid sequence of an extracellular, ligand-binding portion of a receptor A, said receptor A being selected from receptors of the tumor necrosis factor receptor super family (TNFRSF);
a second part comprising an amino acid sequence that is substantially identical to the amino acid sequence of an intracellular, signalling kinase portion of a receptor B, said receptor B being selected from receptor tyrosine kinases (RTKs); and,
between said first and second parts, a third part comprising an amino acid sequence taken from and/or substantially identical to a transmembrane domain.

2. The chimeric polypeptide of claim 1, wherein the amino acid sequence of said first part has the capacity of oligomerization with the corresponding extracellular domain of the receptor A and/or with another chimeric polypeptide of claim 1.

3. The chimeric polypeptide of claim 1, wherein the amino acid sequence of said first part has the capacity of binding an agent exhibiting an activity on receptor A, such as a natural ligand of the receptor A.

4. The chimeric polypeptide of claim 1, wherein the amino acid sequence of said second part has the capacity of oligomerization with the corresponding intracellular domain of the receptor B and/or of another chimeric polypeptide of claim 1.

5. The chimeric polypeptide of claim 1, wherein the amino acid sequence of said second part has tyrosine kinase activity following dimerization.

6. The chimeric polypeptide of claim 1, wherein said transmembrane domain is selected from transmembrane domains of receptors of the TNFRSF and of RTKs.

7. The chimeric polypeptide of claim 1, wherein substantially identical means at least 50%, 60%, 70%, 75%, 80%, 85%, 90%, 95%, 97%, 98%, or 99% amino acid sequence identity with the amino acid of the referred portion and/or stretch.

8. The chimeric polypeptide of claim 1, comprising the full-length amino acid sequence of said receptor A.

9. The chimeric polypeptide of claim 1, wherein the receptor B is selected from the group consisting of platelet derived growth factor receptors (PDGFRs), epidermal growth factor receptors (EGFRs), fibroblast growth factor receptors (FGFR), and vascular endothelial growth factor receptors (VEGFRs).

10. The chimeric polypeptide of claim 1, which comprises an amino acid sequence taken from or substantially identical to a death domain.

11. The chimeric polypeptide of claim 10, wherein said death domain has an amino acid sequence that is substantially identical to the death domain of TNFRSF1.

12. The chimeric polypeptide according to claim 1, which comprises an extracellular, ligand-binding portion of a TNFRSF receptor, a transmembrane domain, and an intracellular, signalling kinase portion of an RTK.

13. A nucleic acid molecule comprising a nucleotide sequence encoding a chimeric polypeptide according to claim 1.

14. A cell expressing the nucleotide sequence as defined in claim 13, and/or in the plasma membrane of which is embedded the encoded chimeric polypeptide.

15. A method of screening agents which are capable of affecting the activity of a receptor A selected from receptors of the tumor necrosis factor receptor super family (TNFRSF), said method comprising the steps of:
providing cells expressing at least one nucleotide sequence encoding a chimeric polypeptide comprising:
a first part comprising an amino acid sequence that is substantially identical to the amino acid sequence of an extracellular, ligand-binding portion of a receptor A, said receptor A being selected from receptors of the tumor necrosis factor receptor super family (TNFRSF);
a second part comprising an amino acid sequence that is substantially identical to the amino acid sequence of an intracellular, signalling kinase portion of a receptor B, said receptor B being selected from receptor tyrosine kinases (RTKs); and,
between said first and second parts, a third part comprising an amino acid sequence taken from and/or substantially identical to a transmembrane domain;
exposing a candidate agent to be screened to said cells;
measuring a physical, biological and/or chemical value that is associated with a cellular condition of said cells; and
determining, from the value measured in the preceding step, if said candidate agent is an agent that is capable of affecting the activity of said receptor A.

16. The method of claim 15, wherein an agent affects the activity of a receptor if it affects a status of signalling of the receptor.

17. The method of claim 15, wherein said candidate is an active agent of said receptor A, if it affects said cellular condition of said cells.

18. The method of claim 15, wherein said cellular condition is at least partly dependent of an activity and/or a condition of said chimeric polypeptide.

19. The method of claim 15, wherein said cellular condition is at least partly dependent of activity or absence of activity of the intracellular kinase domain of said chimeric polypeptide.

20. The method of claim 15, wherein said cellular condition is concentration or a change in the concentration of one or more selected from the group consisting of: intracellular Ca²⁺, inositol phosphate (IP1) and inositol triphosphate (IP3).

21. The method of claim 15, wherein said physical, biological and/or chemical value that is associated with a cellular characteristic is fluorescence or luminescence or both.

22. (canceled)

23. A chimeric polypeptide comprising:
an amino acid sequence that is substantially identical to the amino acid sequence of the extracellular, ligand binding portion of a receptor A, said receptor A being selected from receptors of the TNFRSF,
a transmembrane domain;
optionally, an amino acid sequence that is substantially identical to the amino acid sequence of a death domain; and,
an amino acid sequence that is substantially identical to the amino acid sequence of an intracellular, signalling kinase portion of a receptor B, said receptor B being selected from receptor tyrosine kinases (RTKs).


SEQUENCE LISTING (22 sequences)

SEQ ID NO: 1 - DNA, 2997 bp, Artificial Sequence
  DNA fusion of hTNFR1 full length and mPDGFR cytoplasmic domain
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagacagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 2 - protein, 998 aa, Artificial Sequence
  Protein fusion of hTNFR1 full length and mPDGFR cytoplasmic domain
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLRQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 3 - DNA, 2325 bp, Artificial Sequence
  DNA fusion of hTNFR1 extracellular and tm domain with mPDGFR cytoplasmic domain
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgaagccacgttacgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggatagcttcctgtag

SEQ ID NO: 4 - protein, 774 aa, Artificial Sequence
  Protein fusion of hTNFR1 extracellular and tm domain with mPDGFR cytoplasmic domain
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 5 - DNA, 2349 bp, Artificial Sequence
  DNA fusion of hTNFR1 extracellular with mPDGFR tm and cytoplasmic domain
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccttgccctttaaagtggcggtgatctcagccatcctggccttagtggtccttaccgtcatctctctcatcatcctcatcatgctgtggcagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggatagcttcctgtag

SEQ ID NO: 6 - protein, 782 aa, Artificial Sequence
  Protein fusion of hTNFR1 extracellular with mPDGFR tm and cytoplasmic domain
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTLPFKVAVISAILALVVLTVISLIILIMLWQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 7 - DNA, 2328 bp, Artificial Sequence
  DNA fusion of hTNFR1 extracellular and tm domain with hEGFR cytoplasmic domain
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 8 - protein, 775 aa, Artificial Sequence
  Protein fusion of hTNFR1 extracellular and tm domain with hEGFR cytoplasmic domain
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 9 - DNA, 2328 bp, Artificial Sequence
  DNA fusion of hTNFR1 extracellular domain with hEGFR transmembrane and cytoplasmic domain
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 10 - protein, 775 aa, Artificial Sequence
  Protein fusion of hTNFR1 extracellular domain with hEGFR transmembrane and cytoplasmic domain
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 11 - DNA, 2904 bp, Artificial Sequence
  Fusion hDR3 (fl) - mPDGFR (cp) DNA
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgccacccccgtcccttgcaggagcaccctggggagctgtccagagcgctgtgccgctgtctgtggctggaggcagagtaggtgtgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggatagcttcctgtag

SEQ ID NO: 12 - protein, 967 aa, Artificial Sequence
  Fusion hDR3 - mPDGFR (cp) Prot
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTPPPSLAGAPWGAVQSAVPLSVAGGRVGVFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 13 - DNA, 2202 bp, Artificial Sequence
  DNA fusion of hFAS extracellular and transmembrane domains with mPDGFR cytoplasmic domain
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgcagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcaccttcctgcctctgaagtggatggccccagagagcatcttcaacagcctctacaccattttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 14 - protein, 733 aa, Artificial Sequence
  Protein fusion of hFAS extracellular and transmembrane domains with mPDGFR cytoplasmic domain
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTFLPLKWMAPESIFNSLYTILSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 15 - DNA, 2238 bp, Artificial Sequence
  DNA fusion of hFAS extracellular domain with mPDGFR transmembrane and cytoplasmic domains
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttgccctttaaggtggcggtgatctcagccatcctggccttagtggtccttaccgtcatctctctcatcatcctcatcatgctgtggcagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 16 - protein, 745 aa, Artificial Sequence
  Protein fusion of hFAS extracellular domain with mPDGFR transmembrane and cytoplasmic domains
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLPFKVAVISAILALVVLTVISLIILIMLWQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 17 - DNA, 2937 bp, Artificial Sequence
  DNA fusion of hFAS full-length with hTNFR1 death domain and further with mPDGFR cytoplasmic domain
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagacagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcaccttcctgcctctgaagtggatggccccagagagcatcttcaacagcctctacaccattttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 18 - protein, 978 aa, Artificial Sequence [sequence omitted]

SEQ ID NO: 19 - DNA, 2868 bp, Artificial Sequence
  DNA fusion of hFAS extracellular and transmembrane domains with hTNFR1 cytoplasmic domain and further with mPDGFR cytoplasmic domain
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagacagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcacctacctgcctctgaagtggatggccccagagagcatcttcaacagcctctacaccactttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 20 - protein, 955 aa, Artificial Sequence
  Protein fusion of hFAS extracellular and transmembrane domains with hTNFR1 cytoplasmic domain and further with mPDGFR cytoplasmic domain
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLRQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTYLPLKWMAPESIFNSLYTTLSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL

SEQ ID NO: 21 - DNA, 3369 bp, Artificial Sequence
  DNA fusion of hTNFR2 full length with hTNFR1 death domain and further with mPDGFR cytoplasmic domain
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagtcttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagacagaagaagccacgctatgagatccgatggaaggtcattgagtctgtgagctctgacggtcatgagtacatctacgtggaccctgtgcagttgccttacgactccacctgggagctgccacgggaccagcttgttctgggacgcactcttggctctggggctttcggacaggtggtggaggccacagctcacggtctgagccattcgcaggccaccatgaaagtggctgtcaagatgctgaaatcgacagccagaagtagcgagaagcaagccttaatgtccgagctgaagattatgagtcatcttggaccccacctgaacgtggtcaacctgctgggggcctgcaccaaaggagggcccatctacatcatcacggaatactgccgatacggtgatctggtggactacctgcaccggaacaaacacaccttcttgcagcgacactccaacaagcattgtccgcccagtgctgagctctacagcaacgccctgccagtggggttctccctacccagccacttgaacctgactggggagagtgacggtggctacatggatatgagcaaggatgaatctatagattacgtgcccatgttggacatgaaaggagacatcaaatacgcagacattgagtcccccagctacatggccccttatgataactatgtcccatctgcccctgaaaggacctatcgcgccaccttaatcaacgactcaccagtgctcagctacacagacctcgtgggcttcagctaccaagtggccaacggcatggacttcttagcctctaagaactgtgttcaccgagacttggcggccaggaatgtgctcatctgcgagggcaagctggtcaagatctgtgacttcggcctggctcgagacatcatgagggactcaaactacatctccaaaggcagcaccttcctgcctctgaagtggatggccccagagagcatcttcaacagcctctacaccattttgagtgatgtctggtcttttgggatcctactctgggagatcttcacactgggtggcaccccttacccagagctgcccatgaacgaccagttctacaatgccatcaagaggggctaccgcatggcccagcctgctcatgcctccgacgagatctatgagatcatgcagaaatgctgggaagaaaagtttgagactcgaccccccttctcccagctggtgctgctcctggagaggcttctgggtgaaggctataaaaagaagtaccagcaggtagatgaggagttcctgaggagtgaccatcctgccatcctgaggtcccaagcccgctttccggggatccacagcctccgatcccctctggacaccagctctgttctctacactgccgtgcagcccaatgagagtgacaatgactacatcatccccttacctgaccccaagcctgacgttgctgatgaaggtctcccagaggggtcccccagccttgccagttccaccttgaatgaagtcaacacttcctccaccatctcctgcgacagtcccctggagctccaagaagagccacagcaagcagagcctgaggcacaactggagcagccacaggattcaggctgcccaggacctctggctgaagcagaggacagcttcctgtag

SEQ ID NO: 22 - protein, 1122 aa, Artificial Sequence
  Protein fusion of hTNFR2 full length with hTNFR1 death domain and further with mPDGFR cytoplasmic domain
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLRQKKPRYEIRWKVIESVSSDGHEYIYVDPVQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRHSNKHCPPSAELYSNALPVGFSLPSHLNLTGESDGGYMDMSKDESIDYVPMLDMKGDIKYADIESPSYMAPYDNYVPSAPERTYRATLINDSPVLSYTDLVGFSYQVANGMDFLASKNCVHRDLAARNVLICEGKLVKICDFGLARDIMRDSNYISKGSTFLPLKWMAPESIFNSLYTILSDVWSFGILLWEIFTLGGTPYPELPMNDQFYNAIKRGYRMAQPAHASDEIYEIMQKCWEEKFETRPPFSQLVLLLERLLGEGYKKKYQQVDEEFLRSDHPAILRSQARFPGIHSLRSPLDTSSVLYTAVQPNESDNDYIIPLPDPKPDVADEGLPEGSPSLASSTLNEVNTSSTISCDSPLELQEEPQQAEPEAQLEQPQDSGCPGPLAEAEDSFL